(19) United States
(12) Patent Application Publication
(10) Pub. No.: US 2007/0255243 A1
(43) Pub. Date: Nov. 1, 2007

(54) Dimensionally stable stretchable absorbent composite

(76) Inventors: James Martin Kaun, Neenah, WI (US); Anthony John Wisneski, Kimberly, WI (US); Davis Dang Hoang Nhan, Appleton, WI (US); David Martin Jackson, Alpharetta, GA (US); Kenneth John Zwick, Neenah, WI (US); Patrick Robert Lord, Appleton, WI (US); David Louis Zenker, Neenah, WI (US); Cathleen Mae Uttecht, Menasha, WI (US); Gabriel Hammam Adam, Alpharetta, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/413,444

(22) Filed: Apr. 28, 2006

(51) Int. Cl. A61F 13/15

(52) U.S. Cl. 604/378; 604/385.101; 604/370

(57) ABSTRACT

An absorbent composite has a low capacity region and a high capacity region in planar relationship to the low capacity region, where the high capacity region comprises between 1% and 10% by weight elastomeric polymer fibers and between 60% and 98% by weight superabsorbent material with respect to that region, and where the low capacity region comprises at least 10% by weight elastomeric polymer fibers and less than 10% by weight superabsorbent material. In some embodiments, the absorbent composite can further comprise additional regions. The regions are substantially joined by intermingling of the elastomeric polymer fibers between the regions, to form a generally unitary, stratified absorbent composite. The absorbent composite can be utilized in an absorbent article, the result of which is an absorbent article that exhibits improved performance as well as greater comfort and confidence among the user.

### BACKGROUND

Articles, such as absorbent articles, are useful for absorbing many types of fluids, including fluids secreted or eliminated by the human body. Superabsorbent materials (SAM's) are frequently used in absorbent articles to help improve the absorbent properties of such articles. Superabsorbent materials are generally polymer based and are available in many forms, such as powders, granules, microparticles, films and fibers, for example. Upon contact with fluids, such superabsorbent materials swell by absorbing the fluids into their structures. In general, superabsorbent materials can quickly absorb fluids insulted into such articles, and can retain such fluids to prevent leakage and help provide a dry feel even after fluid insult.

There is a continuing effort to improve the performance of such absorbent articles, especially at high levels of fluid saturation, to thereby reduce the occurrence of leakage and to improve fit and comfort. This is particularly significant when such articles are subjected to repeated fluid insults during use. This has become an increasing challenge as recent efforts in absorbent article design have generally focused on using higher concentrations of superabsorbent material and less fluff fibers to make the absorbent structures thinner and more flexible. However, notwithstanding the increase in total absorbent capacity obtained by increasing the concentration of superabsorbent material, such absorbent articles may still nevertheless leak during use. Such leakage may in part be the result of the absorbent composite component of an article having an insufficient intake rate (i.e., the rate at which a fluid insult can be taken into and entrained within the absorbent composite for subsequent absorption by the superabsorbent material) due to low permeability and lack of available void volume. Therefore, there is a desire for an absorbent article which contains high levels of superabsorbent materials and which maintains a sufficient intake rate.

Conventional absorbent composites are typically not stretchable. However, recent attempts have been made to incorporate elastomeric materials into various structural components of absorbent articles, including the absorbent composite component of such articles, to help achieve better fit, greater comfort, and enhanced containment, as well as sufficient integrity. Adding stretchability to absorbent composites can be difficult because elastomeric materials often are not absorbent, and the addition of elastomeric materials to absorbent composites may inhibit the fluid handling properties of the absorbent composites and have other negative effects. Consequently, stretchable absorbent articles often result in excessive wet growth in the x- and y-planes, poor wet gel containment, decreased superabsorbent capacity efficiency, buckling within the chassis, gasket failure, poor fit and unfavorable perception of the article.

In addition, the use of elastomeric materials in an absorbent article can change the swelling properties of absorbents contained therein. In general, a superabsorbent material is assumed to swell isotropically in isolation. However, this may not be the case once the particle is placed in an absorbent system comprising elastomeric materials because interactions can result in more complex swelling behavior of the system. As the SAM swells, the ability to rearrange within the structure will determine the dimensional changes of that structure. The movement of gel materials within a network is dominated by factors such as swell pressure, friction, and particle interaction. For example, a conventional SAM/fluff absorbent will generally have little dimensional change in either the machine direction (MD) or cross-machine direction (CD). The majority of the expansion is in the z-direction (ZD). In contrast, an absorbent structure stabilized with an elastomeric polymer network imposes restraining forces upon the SAM which can vary in all three axes. Such variations in forces within the elastomeric network are caused by differences in polymer fiber orientation and the manner in which the fibers are mixed and deposited on the forming surface. One characteristic typical of a stretchable structure is that it has a relatively high ZD strength provided by the elastomeric polymer network. Thus, swelling in the ZD is greatly restricted compared to conventional SAM/fluff absorbents. In general, this greater ZD restraint forces higher in-plane expansion. The results are the aforementioned disadvantages.

Thus, there is a need for an article that is both absorbent and stretchable. There is a further need for such an article to be dimensionally stable having improved growth in the ZD while exhibiting the same or reduced growth in the MD and/or CD as compared to conventional stretchable absorbent articles. In addition, there is a need for a stretchable absorbent article to exhibit better wet and/or dry SAM containment as compared to conventional stretchable absorbent articles. There is a further need for a stretchable absorbent article to provide a better fit, particularly after multiple fluid insults, as compared to conventional stretchable absorbent articles.

### SUMMARY

In response to the needs discussed above, an article of the present invention comprises an absorbent composite having a low capacity region, and a high capacity region in planar relationship to the low capacity region, where the high capacity region comprises between 1% and 10%, such as between 1% and 5%, or between 1% and 3% by weight elastomeric polymer fibers and between 60% and 98% by weight superabsorbent material with respect to that region, and where the low capacity region comprises at least 10% by weight elastomeric polymer fibers and less than 10% by weight superabsorbent material. The regions can be substantially joined by intermingling of the elastomeric polymer fibers between the regions, to form a generally unitary (i.e., non-laminated), stratified absorbent composite.

There may be more than one of each region in the composite, and there may be additional regions in the composite. For example, in some aspects, the high capacity region may be sandwiched between a low capacity region and an additional region. In further aspects, the additional region can be another low capacity region which may be the same as, or different from, the first low capacity region. In other aspects, the absorbent composite can have a perimeter area and a central area, where the high capacity region is positioned only in the central area of the absorbent composite. In yet other aspects, the fibers of the low capacity region and the additional region located in the perimeter area are intermingled, which in some features, can form a sealed edge. In some aspects, the sealed edge can encompass one or more portions of the absorbent composite. In other aspects, the sealed edge can compass the entire circumference of the absorbent composite.

In some aspects of the present invention, the elastomeric fibers may be substantially continuous. In addition, the elastomeric fibers can have an average fiber diameter between 5 microns (am) and 50 μm, such as between 10 μm and 25 μm. In other aspects, the low capacity region further comprises between 10% and 90%, such as between 30% and 70%, by weight cellulosic fiber with respect to that region, and may have a basis weight of between 5 and 100 gsm, such as between 10 and 50 gsm. In addition, the low capacity region may comprise substantially meltblown elastomeric polymer fibers. In still other aspects, the high capacity region further comprises 20% or less by weight cellulosic fiber, and can have a basis weight of between 25 and 1000 gsm. In particular features, at least one of the regions can be treated to be hydrophilic.

In some features, the absorbent composite may include at least 40% by weight superabsorbent material, such as at least 60% by weight, or between 60% and 95% by weight superabsorbent material. In addition, at least a portion of the superabsorbent material can comprise a coating to improve attachment of the superabsorbent material to the elastomeric polymer fibers when compared to an uncoated superabsorbent material.

In some aspects, the absorbent composite can have an absorbency of at least 16 g/g as measured by the Saturated Capacity Test. In other aspects, the absorbent composite can have a fluid intake rate of at least 0.4 ml/sec as measured by the Fluid Intake Rate Test. In still another aspect, the absorbent composite can have a Geometric Mean Growth of 20% or less as measured by the Geometric Mean Growth Test. In yet another aspect, the absorbent composite can have a Geometric Mean Growth of 10% or less as measured by the Geometric Mean Growth Test.

In some aspects, the absorbent composite can have an MD Modulus at least 75 times greater, such as 100 times greater, than the ZD tensile strength as measured by the MD Modulus Test and the ZD Tensile Test, respectively. In other aspects, the absorbent composite can have a geometric mean modulus of less than 1 MPa as measured by the Geometric Mean Modulus Test. In yet another aspect, the absorbent composite can have an MD elongation at 50% extension of at least 100 gram-force/inch (g_{f}/inch). In still another aspect, the absorbent composite can have a CD elongation at 50% extension of at least 100 g_{f}/inch.

In some features, the absorbent composite may further include a liquid-permeable topsheet, a backsheet, or both. In addition, the stratified absorbent composite may be disposed between and may be in facing relationship with the topsheet and/or backsheet.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention.

### FIGURES

The foregoing and other features, aspects and advantages of the present invention will become better understood with regard to the following description, appended claims and accompanying drawings where:

FIGS. 1A and 1B are a cross-section of an absorbent composite of the present invention having a low capacity region and a high capacity region;

FIGS. 2A and 2B are a cross-section of an absorbent composite of the present invention having a high capacity region positioned between a low capacity region and an additional region;

FIG. 3 is a cross-section of a stratified absorbent composite of the present invention having a perimeter area that is substantially joined;

FIG. 4 is a schematic diagram of one version of a method and apparatus for producing an absorbent composite of the present invention;

FIG. 5 is a top perspective view of one version of a method and apparatus for producing an absorbent composite of the present invention;

FIG. 6 is a perspective view of one embodiment of an absorbent article that may be made in accordance with the present invention;

FIG. 7 is a plan view of the absorbent article shown in FIG. 6 with the article in an unfastened, unfolded and laid flat condition showing the surface of the article that faces the wearer when worn and with portions cut away to show underlying features;

FIG. 8A is a cross-section side view of an absorbent bandage of the present invention;

FIG. 8B is a top perspective view of an absorbent bandage of the present invention;

FIG. 9 is a top perspective view of an absorbent bed or furniture liner of the present invention;

FIG. 10 is a perspective view of an absorbent sweatband of the present invention;

FIG. 11 is a graphical illustration of the in-plane wet growth properties of the invention compared to various homogeneous absorbents using the Full Pad Growth Test;

FIG. 12 is a partially cut away top view of a Saturated Capacity tester;

FIG. 13 is a side view of a Saturated Capacity tester;

FIG. 14 is a rear view of a Saturated Capacity tester;

FIG. 15 is a top view of the test apparatus employed for the Fluid Intake Rate Test; and

FIG. 16 is a side view of the test apparatus employed for the Fluid Intake Rate Test.

Repeated use of reference characters in the present specification and drawings is intended to represent the same or analogous features or elements of the present invention.

### DEFINITIONS

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The term “absorbent article” generally refers to devices which can absorb and contain fluids. For example, personal care absorbent articles refer to devices which are placed against or near the skin to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, personal care absorbent articles, health/medical absorbent articles, household/industrial absorbent articles, and sports accessory absorbent articles.

The term “coform” is intended to describe a blend of meltblown fibers and cellulose fibers that is formed by air forming a meltblown polymer material while simultaneously blowing air-suspended cellulose fibers into the stream of meltblown fibers. The coform material may also include other materials, such as superabsorbent materials. The meltblown fibers containing wood fibers and/or other materials are collected on a forming surface, such as provided by a foraminous belt. The forming surface may include a gas-pervious material, such as spunbonded fabric material, that has been placed onto the forming surface.

The terms “elastic,” “elastomeric,” “elastically” and “elastically extensible” are used interchangeably to refer to a material or composite that generally exhibits properties which approximate the properties of natural rubber. The elastomeric material is generally capable of being extended or otherwise deformed, and then recovering a significant portion of its shape after the extension or deforming force is removed.

The term “extensible” refers to a material that is generally capable of being extended or otherwise deformed, but which does not recover a significant portion of its shape after the extension or deforming force is removed.

The term “fiber diameter” is the average fiber diameter measured from a sufficient sample size of melt blown fibers or fiber segments to result in a relatively stable mean. Manual or automated measurement techniques can be used to acquire the fiber values.

The term “fluid impermeable,” when used to describe a layer or laminate, means that fluids, such as water or bodily fluids, will not pass substantially through the layer or laminate under ordinary use conditions in a direction generally perpendicular to the plane of the layer or laminate at the point of fluid contact.

The term “health/medical absorbent articles” includes a variety of professional and consumer health-care products including, but not limited to, products for applying hot or cold therapy, medical gowns (i.e., protective and/or surgical gowns), surgical drapes, caps, gloves, face masks, bandages, wound dressings, wipes, covers, containers, filters, disposable garments and bed pads, medical absorbent garments, underpads, and the like.

The term “household/industrial absorbent articles” includes construction and packaging supplies, products for cleaning and disinfecting, wipes, covers, filters, towels, disposable cutting sheets, bath tissue, facial tissue, nonwoven roll goods, home-comfort products including pillows, pads, mats, cushions, masks and body care products such as products used to cleanse or treat the skin, laboratory coats, coveralls, trash bags, stain removers, topical compositions, pet care absorbent liners, laundry soil/ink absorbers, detergent agglomerators, lipophilic fluid separators, and the like.

The terms “hydrophilic” and “wettable” are used interchangeably to refer to a material having a contact angle of water in air of less than 90 degrees. The term “hydrophobic” refers to a material having a contact angle of water in air of at least 90 degrees. For the purposes of this application, contact angle measurements are determined as set forth in Robert J. Good and Robert J. Stromberg, Ed., in “Surface and Colloid Science—Experimental Methods,” Vol. 11, (Plenum Press, 1979), which is hereby incorporated by reference in a manner that is consistent herewith.

The term “Interpenetrating Polymer Network” (IPN) is an important interfacial structure between two polymers which can help enhance bonding integrity between a superabsorbent material and other components of an absorbent composite. IPN pertains to macromolecular chains of a polymer which penetrate through the interface into another polymer domain, or vice versa. Such a penetrating network can promote bond strength, and typically occurs only between compatible polymers. The process employed to coat one polymer onto the other may affect the formation of the desired IPN structure. For example, when a thermally processible and water-soluble polymer (e.g., a hydroxypropyl cellulose, HPC, or a polyethylene oxide, PEO) is coated or otherwise applied onto a base superabsorbent polymer (e.g., a crosslinked sodium polyacrylate), there are two primary coating techniques. One application technique is to spray fine droplets of molten HPC or PEO onto the surface of superabsorbent material. A second technique is to dissolve the HPC or PEO into water to form a solution, and then mix the solution with dry superabsorbent material to allow the material to absorb the solution. The first technique typically produces a coating with no IPN formation. The second technique can promote the formation of the IPN at the interface between the superabsorbent material and the surface coating material due to a swelling of the superabsorbent, and a diffusion and penetration of water molecules into superabsorbent material during the operation of the coating technique

The term “layer” when used in the singular can have the dual meaning of a single element or a plurality of elements.

The term “material” when used in the phrase “superabsorbent material” refers generally to discrete units. The units can comprise particles, granules, fibers, flakes, agglomerates, rods, spheres, needles, particles coated with fibers or other additives, pulverized materials, powders, films, and the like, as well as combinations thereof. The materials can have any desired shape such as, for example, cubic, rod-like, polyhedral, spherical or semi-spherical, rounded or semi-rounded, angular, irregular, etc. Additionally, superabsorbent material may be composed of more than one type of material.

The term “MD” or “machine direction” refers to the orientation of the absorbent web that is parallel to the running direction of the forming fabric and generally within the plane formed by the forming surface. The term “CD” or “cross-machine direction” refers to the direction perpendicular to the MD and generally within the plane formed by the forming surface. Both MD and CD generally define a plane that is parallel to the forming surface. The term “ZD” or “Z-direction” refers to the orientation that is perpendicular to the plane formed by the MD and CD.

The term “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. In the particular case of a coform process, the meltblown fiber stream intersects with one or more material streams that are introduced from a different direction. Thereafter, the meltblown fibers and other materials are carried by the high velocity gas stream and are deposited on a collecting surface. The distribution and orientation of the meltblown fibers within the formed web is dependent on the geometry and process conditions. Under certain process and equipment conditions, the resulting fibers can be substantially “continuous,” defined as having few separations, broken fibers or tapered ends when multiple fields of view are examined through a microscope at 10× or 20× magnification. When “continuous” melt blown fibers are produced, the sides of individual fibers will generally be parallel with minimal variation in fiber diameter within an individual fiber length. In contrast, under other conditions, the fibers can be overdrawn and strands can be broken and form a series of irregular, discrete fiber lengths and numerous broken ends. Retraction of the once attenuated broken fiber will often result in large clumps of polymer.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded-carded-web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

The terms “particle,” “particles,” “particulate,” “particulates” and the like, when used with the term “superabsorbent” or “superabsorbent polymer” refers to the form of discrete units. The units can comprise flakes, fibers, agglomerates, granules, powders, spheres, pulverized materials or the like, as well as combinations thereof. The particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers, are also contemplated for inclusion herein. The terms “particle” or “particulate” may also include an agglomeration comprising more than one individual particle, particulate or the like. Additionally, a particle, particulate or any desired agglomeration thereof may be composed of more than one type of material.

The term “personal care absorbent article” includes, but is not limited to, absorbent articles such as diapers, diaper pants, baby wipes, training pants, absorbent underpants, child care pants, swimwear, and other disposable garments; feminine care products including sanitary napkins, wipes, menstrual pads, menstrual pants, panty liners, panty shields, interlabials, tampons, and tampon applicators; adult-care products including wipes, pads such as breast pads, containers, incontinence products, and urinary shields; clothing components; bibs; athletic and recreation products; and the like.

The term “polymers” includes, but is not limited to, homopolymers, copolymers, such as for example, block, graft, random and alternating copolymers, terpolymers, etc. and blends and modifications thereof. Furthermore, unless otherwise specifically limited, the term “polymer” shall include all possible configurational isomers of the material. These configurations include, but are not limited to isotactic, syndiotactic and atactic symmetries.

The term “polyolefin” as used herein generally includes, but is not limited to, materials such as polyethylene, polypropylene, polyisobutylene, polystyrene, ethylene vinyl acetate copolymer and the like, the homopolymers, copolymers, terpolymers, etc., thereof, and blends and modifications thereof. The term “polyolefin” shall include all possible structures thereof, which includes, but is not limited to, isotatic, synodiotactic and random symmetries. Copolymers include random and block copolymers.

The term “sports accessory absorbent articles” includes headbands, wrist bands and other aids for absorption of perspiration, absorptive windings for grips and handles of sports equipment, and towels or absorbent wipes for cleaning and drying off equipment during use.

The terms “spunbond” and “spunbonded fiber” refer to fibers which are formed by extruding filaments of molten thermoplastic material from a plurality of fine, usually circular, capillaries of a spinneret, and then rapidly reducing the diameter of the extruded filaments.

The term “stratified” refers to having essentially no discernable boundary between regions in the absorbent composite of the present invention to form a generally unitary (i.e., non-laminated) composite. One way to accomplish this is by entangling the elastomeric polymer fibers between the regions of the absorbent composite, as well as by polymeric bonding of the fibers. Although generally unitary, a stratified composite can exhibit structural differences in composition in the Z-direction used to impart different properties and functionality of the regions achieved during deposition of the materials being used. In contrast, the term “layered” refers to having a definite discernable boundary between layers of an absorbent composite, such as would be found in a laminated absorbent composite, for example. For evaluation purposes during manufacture, each region of the stratified absorbent composite can be produced separately and then tested to determine relevant properties associated with each particular region. For finished composites possessing multiple regions, each region can be identified by using sectioning techniques well known in the art followed by the appropriate analytical testing for composition and performance properties.

The term “stretchable” refers to materials which may be extensible or which may be elastically extensible.

The terms “superabsorbent” and “superabsorbent material” refer to water-swellable, water-insoluble organic or inorganic materials capable, under the most favorable conditions, of absorbing at least about 10 times their weight, or at least about 15 times their weight, or at least about 25 times their weight in an aqueous solution containing 0.9 weight percent sodium chloride. In contrast, “absorbent materials” are capable, under the most favorable conditions, of absorbing at least 5 times their weight of an aqueous solution containing 0.9 weight percent sodium chloride.

The term “target zone” refers to an area of an absorbent composite where it is particularly desirable for the majority of a fluid insult, such as urine, menses, or bowel movement, to initially contact. In particular, for an absorbent composite with one or more fluid insult points in use, the insult target zone refers to the area of the absorbent composite extending a distance equal to 15% of the total length of the composite from each insult point in both directions.

The term “thermoplastic” describes a material that softens when exposed to heat and which substantially returns to a non-softened condition when cooled to room temperature.

These terms may be defined with additional language in the remaining portions of the specification.

### DETAILED DESCRIPTION

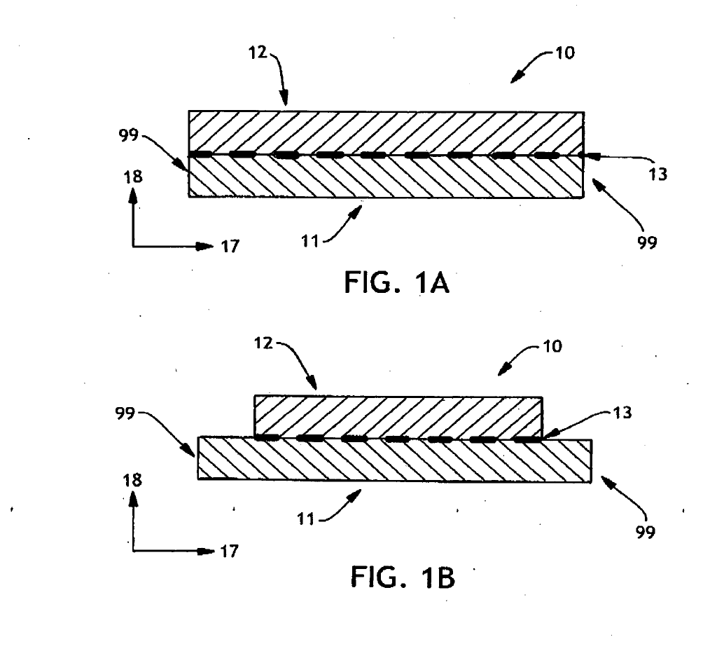

The absorbent composite of the present invention comprises a high capacity region and a low capacity region. To gain a better understanding of the present invention, attention is directed to FIG. 1A, which shows a cross-section of the absorbent composite of the present invention. The absorbent composite has a machine direction (MD) (not shown), a cross-machine direction (CD) 17 and a Z-direction (ZD) 18.

In FIG. 1A, the absorbent composite 10 has a low absorbent capacity region 11 and a high absorbent capacity region 12. The regions are configured such that the absorbent composite 10 is stratified rather than layered as defined above, generally shown by broken line 13. Attachment between the low capacity region 11 and the high capacity region 12 will generally be continuous along broken line 13 in the form of intermingled fibers or bonding between the polymer fibers that occurs as the regions are formed. The fiber entanglement or polymeric bonding along broken line 13 is generally indistinguishable from that found in the absorbent matrix of either region. Although there will be a change in composition in the various components in the ZD as one passes from one region to the next in the ZD, there will be essentially no discernable boundary between the regions. In addition, the change in concentration of the various components in each region can range from being fairly distinct to appearing as a gradient, depending on the characteristics desired and on the process used to produce the absorbent.

As is shown in FIG 1A, the high capacity region 12 is coextensive with the low capacity region 11. However, in the present invention, it is not necessary that the high capacity region 12 is coextensive with the low capacity region 11. That is, the high capacity region 12 may not completely cover the low capacity region 11 to the outer edges 99 of the low capacity region 11. In an alternative embodiment of the present invention shown in FIG. 1B, the high capacity region 12 is not coextensive with the low capacity region 11, covering only a portion of the low capacity region 11 short of the outer edges 99 of the low capacity region 11. In some aspects of the invention, the low capacity region 11 acts as a support layer, supporting the high capacity region 12.

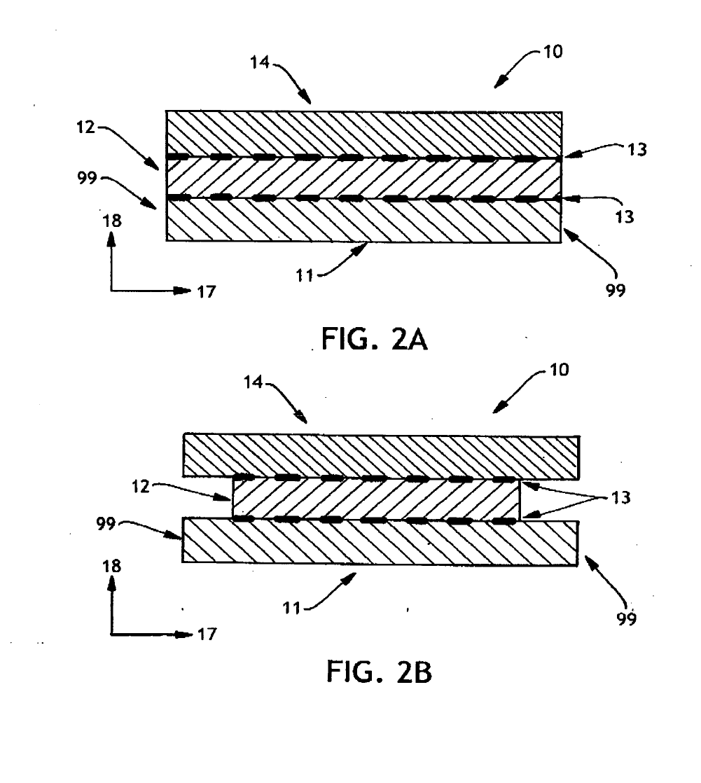

In other features, the absorbent composite 10 can have any number of additional regions. To obtain a better understanding of this aspect of the present invention, attention is directed to FIG. 2A, which shows an exemplary absorbent composite 10 having a high capacity region 12 sandwiched between a low capacity region 11 and an additional region 14. The absorbent composite has a MD (not shown), a CD 17 and a ZD 18. In some aspects, the additional region 14 may be a second low capacity region, which may or may not be the same as the first low capacity region 11. The regions are configured such that the absorbent composite 10 is stratified rather than layered, shown by the broken line 13.

As is shown in FIG. 2A, the high capacity region 12 is coextensive with the low capacity region 11 and the additional region 14. However, in the present invention, it is not necessary that the high capacity region 12 is coextensive with the low capacity region 11 and the additional region 14. That is, the high capacity region 12 may not reach the outer edges 99 of the low capacity region 11 and the additional region 14. In an alternative embodiment of the present invention shown in FIG. 2B, the high capacity region 12 is not coextensive with the low capacity region 11 and the additional region 14, covering only a portion of the low capacity region 11 and the additional region 14 short of the outer edges 99.

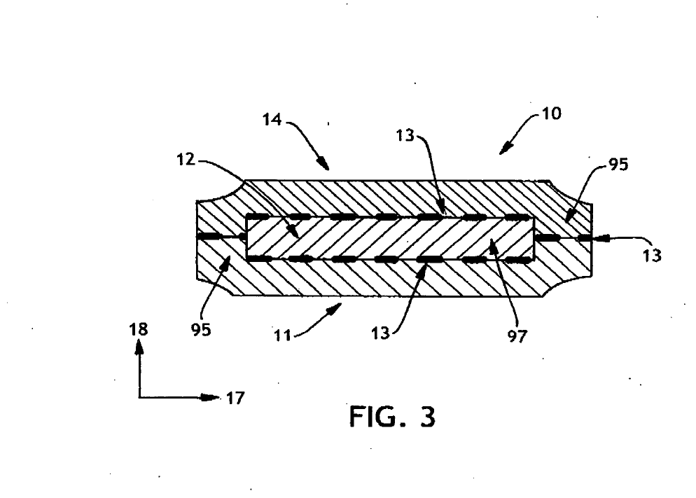

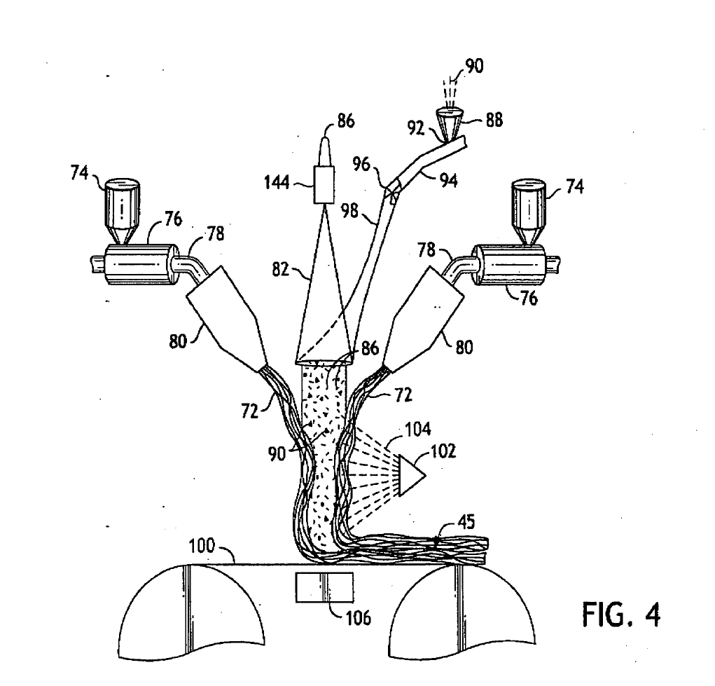

In some aspects of the invention, the absorbent composite may have two distinct areas of the composite which have different stratified regions. To obtain a better understanding of this aspect of the present invention, attention is directed to FIG. 3, which shows an absorbent composite 10 having a central area 97 and a perimeter area 95. The absorbent composite has a MD (not shown), a CD 17 and a ZD 18. The central area includes both a low capacity region 11, an additional region 14 and a high capacity region 12, which are stratified as shown by broken line 13. The perimeter area 95 only includes the low capacity region 11 and the additional region 14, which also are stratified as shown by the broken line 13. The perimeter area of this embodiment can thus form a sealed edge. For purposes of this invention, the term “perimeter” does not necessarily form a closed loop around an absorbent composite, but rather merely refers to an edge portion of the composite. Thus, an absorbent composite of the present invention may have more than one perimeter area.

In particular aspects, the absorbent composite can include stratified regions which comprise varying amounts of absorbent and/or elastomeric material. For example, the absorbent composite can have at least one low capacity region and at least one high capacity region. In one particular feature, the high capacity region comprises elastomeric polymer fibers in a certain concentration and a superabsorbent material in a certain concentration. The material distribution within each region may or may not be homogeneous. In some aspects, the material distribution forms a gradient.

In some aspects, the high capacity region may comprise elastomeric polymer fibers in a concentration of about 10% or less by weight in that region, such as about 5% or less, or about 3% or less, or between about 1% and 10%, or between about 1% and 5% or between about 1% or 3% by weight with respect to that region. The high capacity region further comprises superabsorbent material in a concentration of about 60% or greater by weight in that region, such as about 70% or greater, or about 80% or greater, or about 90% or greater or between about 60% and 98% by weight with respect to that region. In addition, the low capacity region 11 may comprise elastomeric polymer fibers in a concentration of about 10% or greater by weight in that region, such as about 70% or greater or about 90% or greater by weight.

If the amount of elastomeric polymer in each region of the absorbent composite is outside the desired values, various disadvantages can occur. For example, an insufficient amount of elastomeric polymer may provide an inadequate level of structural integrity, and an inadequate ability to stretch and retract elastically. An excessively high amount of elastomeric polymer in a region intended for absorption may hold superabsorbent material too tightly and may inhibit the swelling of superabsorbent. In this scenario, the restricted swelling of the superabsorbent material can excessively limit the absorbent capacity of the composite. Where the elastomeric polymer is generally hydrophobic, an excessively large amount of elastomeric polymer in a region intended for absorption may undesirably limit the intake rate at which the composite acquires fluid, and may limit the distribution of fluid to other parts of the absorbent composite. Furthermore, an excessive amount of elastomeric polymer may hinder the ability of the absorbent composite to stretch in that region, particularly in the ZD. Alternatively, an insufficient amount of elastomeric fibers in a region may result in poor superabsorbent material containment, cracking and buckling, which in turn can result in poor fit and discomfort.

It has been found that certain relationships between particular properties imparted by the two or more regions of the absorbent composite will yield unexpected benefits when the regions are present together in a single absorbent. For example, a comparatively low elastomeric polymer content in the high capacity region reduces the restraining forces acting upon superabsorbent material attempting to swell in the ZD. The polymer content in the low capacity region provides restraining forces that can counteract the in-plane expansive forces exerted by the high absorbent region. Together, the two regions provide an absorbent that allows improved ZD swelling while limiting the undesirable in-plane growth common to conventional stretchable absorbent composites.

This relationship can be expressed mathematically. An absorbent structure stabilized with an elastomeric polymer network has restraining forces within the structure in the MD, CD and ZD. Analyses of such structures suggest the swelling in the MD-CD plane (x-y plane) may be described by the expression:

$${ɛ}_{GM} = \frac{{E}_{ZD}}{{E}_{GM}} {ɛ}_{ZD} = \frac{{σ}_{ZD}}{{E}_{GM}}$$

where ε_{GM} is the swelling (or strain) in the plane, E_{ZD} is the ZD modulus, E_{GM} is the in-plane modulus, ε_{ZD} is the ZD swelling, and σ_{ZD} is the ZD strength which is equal to the maximum value of the Z direction swelling times the ZD modulus and gives the maximum ZD restraining force against swelling.

When an elastomeric absorbent is used for wound care for example, such as in a bandage or compress, and especially in applications to contain high seepage, a high degree of dimensional stability is required throughout the period of use. A conformable fit under all conditions is needed to maintain a seal on the wound to prevent contamination and to prevent leakage of bodily fluids. In this particular aspect, the invention seeks to provide a highly absorbent, close fitting bandage that maintains its coverage and performance under high amounts of liquid loading.

The elastomeric material of the polymer fibers may include an olefin elastomer or a non-olefin elastomer, as desired. For example, the elastomeric fibers can include olefinic copolymers, polyethylene elastomers, polypropylene elastomers, polyester elastomers, polyisoprene, cross-linked polybutadiene, diblock, triblock, tetrablock, or other multi-block thermoplastic elastomeric and/or flexible copolymers such as block copolymers including hydrogenated butadiene-isoprene-butadiene block copolymers; stereoblock polypropylenes; graft copolymers, including ethylene-propylene-diene terpolymer or ethylene-propylene-diene monomer (EPDM) rubber, ethylene-propylene random copolymers (EPM), ethylene propylene rubbers (EPR), ethylene vinyl acetate (EVA), and ethylene-methyl acrylate (EMA); and styrenic block copolymers including diblock and triblock copolymers such as styrene-isoprene-styrene (SIS), styrene-butadiene-styrene (SBS), styrene-isoprene-butadiene-styrene (SIBS), styrene-ethylene/butylene-styrene (SEBS), or styrene-ethylene/propylene-styrene (SEPS), which may be obtained from Kraton Inc. under the trade designation KRATON elastomeric resin or from Dexco, a division of ExxonMobil Chemical Company under the trade designation VECTOR (SIS and SBS polymers); blends of thermoplastic elastomers with dynamic vulcanized elastomer-thermoplastic blends; thermoplastic polyether ester elastomers; ionomeric thermoplastic elastomers; thermoplastic elastic polyurethanes, including those available from Invista Corporation under the trade name LYCRA polyurethane, and ESTANE available from Noveon, Inc., a business having offices located in Cleveland, Ohio U.S.A.; thermoplastic elastic polyamides, including polyether block amides available from AtoFina Chemicals, Inc. (a business having offices located in Philadelphia, Pa. U.S.A.) under the trade name PEBAX; polyether block amide; thermoplastic elastic polyesters, including those available from E. I. Du Pont de Nemours Co., under the trade name HYTREL, and ARNITEL from DSM Engineering Plastics (a business having offices located in Evansville, Ind., U.S.A.) and single-site or metallocene-catalyzed polyolefins having a density of less than about 0.89 grams/cubic centimeter, available from Dow Chemical Co. (a business having offices located in Freeport, Tex. U.S.A.) under the trade name AFFINITY; and combinations thereof.

As used herein, a tri-block copolymer has an ABA structure where the A represents several repeat units of type A, and B represents several repeat units of type B. As mentioned above, several examples of styrenic block copolymers are SBS, SIS, SIBS, SEBS and SEPS. In these copolymers the A blocks are polystyrene and the B blocks are a rubbery component. Generally, these triblock copolymers have molecular weights that can vary from the low thousands to hundreds of thousands, and the styrene content can range from 5% to 75% based on the weight of the triblock copolymer. A diblock copolymer is similar to the triblock, but is of an AB structure. Suitable diblocks include styrene-isoprene diblocks, which have a molecular weight of approximately one-half of the triblock molecular weight having the same ratio of A blocks to B blocks.

In desired arrangements, the polymer fibers can include at least one material selected from the group consisting of styrenic block copolymers, elastic polyolefin polymers and co-polymers and EVA/EMA type polymers.

In some particular arrangements, for example, the elastomeric material of the polymer fibers can include various commercial grades of low crystallinity, lower molecular weight metallocene polyolefins, available from ExxonMobil Chemical Company (a company having offices located in Houston, Tex., U.S.A.) under the VISTAMAXX trade designation. Some VISTAMAXX materials are believed to be metallocene propylene ethylene co-polymer. For example, in one aspect the elastomeric polymer can be VISTAMAXX VM 2210. In other aspects, the elastomeric polymer can be VISTAMAXX PLTD 1778. In one particular aspect, the elastomeric polymer is VISTAMAXX VM 2370. Another optional elastomeric polymer is KRATON blend G 2755 from Kraton Inc. The KRATON material is believed to be a blend of styrene ethylene-butylene styrene polymer, ethylene waxes and tackifying resins.

In some aspects, the elastomeric polymer fibers may be substantially continuous. In further aspects, the elastomeric polymer fibers are provided using a meltblown process, a coform process, and the like. It should be understood that other processes known in the art may also be utilized without departing from the scope of the invention.

The elastomeric polymer fibers may have desirable fiber diameters. In a particular feature, an operative amount of the elastomeric polymer fibers can have an average fiber diameter of not less than about 5 microns (μm), such as not less than about 10 μm. Another feature can have a configuration in which an operative amount of the polymer fibers have an average fiber diameter of not greater than about 50 μm, such as not greater than 25 μm.

If the average fiber diameter is less than 5 μm, the absorbent composite may exhibit inadequate levels of stretchability. An overly great amount of the small polymer fibers in a region intended for absorption, such as in a high capacity region, may also excessively constrain the superabsorbent material and not allow a desired amount of swelling in the superabsorbent material, and could furthermore create a low permeability network that limits fluid intake rates. Additionally, the smaller fibers can become stress crystallized, and the tensions (modulus) of the stretchable composite can be too high.

If the average fiber diameter is greater than 50 microns, the absorbent composite may exhibit inadequate levels of material containment. For a given amount of polymer, such relatively coarse elastomeric fibers may not provide a sufficient amount of fiber surface area, and superabsorbent material may not be adequately contained and held in the matrix of elastomeric polymer fibers.

In some aspects, the elastomeric polymer fibers can be produced from a polymer material having a selected melt flow rate (MFR). In some aspects, the MFR can be up to a maximum of about 300. Alternatively, the MFR can be up to about 230 or 250. In other aspects, the MFR can be a minimum of not less than about 9, or not less than about 20. The MFR can alternatively be not less than about 60 to provide desired performance. The described melt flow rate has the units of grams flow per 10 minutes (g/IO min). The parameter of melt flow rate is well known, and can be determined by conventional techniques, such as by employing test ASTM D 1238 70 “extrusion plastometer” Standard Condition “L” at 230° C. and 2.16 kg applied force.

In another feature, the elastomeric polymer fibers can include an operative amount of a surfactant, which can increase the hydrophilicity of a region. The surfactant can be combined with the polymer fibers in any operative manner. Various techniques for combining the surfactant are conventional and well known to persons skilled in the art. For example, the surfactant may be compounded with the polymer employed to form the meltblown fibers. In a particular feature, the surfactant may be configured to operatively migrate or segregate to the outer surface of the fibers upon the cooling of the fibers. Alternatively, the surfactant may be applied to or otherwise combined with the polymer fibers after the fibers have been formed.

The polymer fibers can include an operative amount of a surfactant, based on the total weight of the fibers and surfactant. In particular aspects, the polymer fibers can include at least a minimum of about 0.1% by weight surfactant, as determined by water extraction. The amount of surfactant can alternatively be at least about 0.15% by weight, and can optionally be at least about 0.2% by weight to provide desired benefits. In other aspects, the amount of surfactant can be generally not more than a maximum of about 2% by weight, such as not more than about 1% by weight, or not more than about 0.5% by weight to provide improved performance.

If the amount of surfactant is outside the desired ranges, various disadvantages can occur. For example, an excessively low amount of surfactant may not allow the hydrophobic meltblown fibers to wet with the absorbed fluid. An excessively high amount of surfactant may allow the surfactant to wash off from the fibers and undesirably interfere with the ability of the composite to transport fluid, or may adversely affect the attachment strength of the absorbent composite to an absorbent article. Where the surfactant is compounded or otherwise internally added to the elastomeric polymer, an excessively high level of surfactant can create conditions that cause a poor formation of the polymer fibers. Processibility suffers as well at high surfactant levels resulting in slippage within the extruder of a meltblown process and loss of polymer pressure and throughput.

In desired configurations, the surfactant can include at least one material selected from the group that includes polyethylene glycol ester condensates and alkyl glycoside surfactants. For example, the surfactant can be a GLUCOPON surfactant, available from Cognis Corporation, a business having offices located in Cincinnati, Ohio, U.S.A., which can be composed of 40% water, and 60% d-glucose, decyl, octyl ethers and oligomerics.

A particular example of a sprayed-on surfactant can include a water/surfactant solution which includes 16 liters of hot water (about 45° C. to 50° C.) mixed with 0.20 kg of GLUCOPON 220 UP surfactant and 0.36 kg of ALCHOVEL Base N-62 surfactant. This is a 1:3 ratio of the GLUCOPON 220 UP surfactant to the ALCHOVEL Base N-62 surfactant. GLUCOPON 220 UP is available from Cognis Corporation, a business having offices located in Cincinnati, Ohio, U.S.A. ALCHOVEL Base-N62 is available from Uniqema, a business having offices located in New Castle, Del., U.S.A. When employing a sprayed-on surfactant, a relatively lower amount of sprayed-on surfactant may be desirable to provide the desired containment of the superabsorbent material. Excessive amounts of the fluid surfactant may hinder the desired attachment of the superabsorbent material to the molten, elastomeric meltblown fibers.

An example of an internal surfactant or wetting agent that can be compounded with the elastomeric fiber polymer can include a MAPEG DO 400 PEG (polyethylene glycol) ester. This material is available from BASF, a business having offices located in Freeport, Tex., U.S.A. Other internal surfactants can include a polyether, a fatty acid ester, a soap or the like, as well as combinations thereof. In one example, IRGASURF HL 560 from Ciba Specialty Chemicals (having a place of business in Tarrytown, N.Y., U.S.A.) was utilized at an addition level of 1.5% by weight.

The absorbent composite of the present invention may also comprise natural fibers, such as cellulosic fibers. In some aspects, the low capacity region can comprise about 90% or less by weight cellulosic fiber, such as about 70% or less, or between about 10% and 90%, or between 30% and 70% by weight with respect to that region. In other aspects, the high capacity region can comprise about 20% or less by weight cellulosic fiber.

The selected amounts of cellulosic or other hydrophilic fiber can help provide increased levels of fluid intake and wicking. Excessive amounts of hydrophilic fibers, however, can undesirably increase the caliper of the composite and may limit properties such as elasticity, stretch and recovery. Additionally, overly large amounts of the hydrophilic fiber can lead to excessive cracking of the absorbent composite during extension and stretching. Large amounts of cellulosic fibers that result in excessively high fluff concentrations may also increase flocculation and result in inferior formation.

The cellulosic fibers may include, but are not limited to, chemical wood pulps such as sulfite and sulfate (sometimes called Kraft) pulps, as well as mechanical pulps such as ground wood, thermomechanical pulp and chemithermomechanical pulp. More particularly, the pulp fibers may include cotton, typical wood pulps, cellulose acetate, rayon, thermomechanical wood pulp, chemical wood pulp, debonded chemical wood pulp, milkweed floss, and combinations thereof.

Pulps derived from both deciduous and coniferous trees can be used. Additionally, the cellulosic fibers may include such hydrophilic materials as natural plant fibers, cotton fibers, microcrystalline cellulose, microfibrillated cellulose, or any of these materials in combination with wood pulp fibers. Suitable cellulosic fibers can include, for example, NB 416, a bleached southern softwood Kraft pulp, available from Weyerhaeuser Co., a business having offices located in Federal Way, Wash. U.S.A.; CR 54, a bleached southern softwood Kraft pulp, available from Bowater Inc., a business having offices located in Greenville, S.C. U.S.A.; SULPHATATE HJ, a chemically modified hardwood pulp, available from Rayonier Inc., a business having offices located in Jesup Ga. U.S.A.; CF405, a chemically treated fluff pulp, available from Weyerhaeuser Co.; and CR 1654, a mixed bleached southern softwood and hardwood Kraft pulp, available from Bowater Inc. In one example, CF405 was utilized.

The absorbent composite of the present invention also includes superabsorbent material. In some features, the absorbent composite may include at least 40% by weight superabsorbent material, such as at least 60% by weight, or at least 70% by weight, or at least 80% by weight, or at least 90% by weight, or between 60% and 95% by weight superabsorbent material. More specifically, the high capacity region can include between 60% and 98% by weight superabsorbent material with respect to that region, and the low capacity region can include less than 10% by weight superabsorbent material with respect to that particular region.

The superabsorbent material can be selected from natural, synthetic and modified natural polymers and materials. The superabsorbent material can be inorganic materials, such as silica gels, or organic compounds, such as crosslinked polymers. The term “crosslinked” refers to any means for effectively rendering normally water-soluble materials substantially water insoluble, but swellable. Such means can comprise, for example, physical entanglement, crystalline domains, covalent bonds, ionic complexes and associations, hydrophilic associations, such as hydrogen bonding, and hydrophobic associations or Van der Waals forces.

Examples of synthetic polymeric superabsorbent materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent composite include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, issued to Masuda et al., and U.S. Pat. No. 4,286,082, issued to Tsubakimoto et al., all of which are incorporated herein by reference in a manner that is consistent herewith.

The superabsorbent material may be in a variety of geometric forms. In one example, the superabsorbent material is in the form of discrete particles. However, the superabsorbent material may also be in the form of fibers, flakes, rods, spheres, needles, particles coated with fibers or other additives, films, and the like, as described above.

Superabsorbent materials suitable for use in the present invention are known to those skilled in the art. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, chitosan salt, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyvinyl amines, polyquatemary ammonium, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are desirably lightly crosslinked to render the material substantially water insoluble. Crosslinking may, for example, be by irradiation or covalent, ionic, Van der Waals, or hydrogen bonding. Suitable base superabsorbent materials are available from various commercial vendors, such as Stockhausen, Inc., BASF Inc. and others. In one example, the superabsorbent material was SR 1642, available from Stockhausen, Inc., a business having offices located in Greensboro, N.C., U.S.A.

The superabsorbent material may desirably be included in an appointed storage or retention portion of the absorbent system, and may optionally be employed in other components or portions of the absorbent article. In one feature, the superabsorbent material can be selectively positioned within the composite such that the absorbent composite comprises regions of varying superabsorbent material concentration. Superabsorbent materials can be incorporated into the absorbent composite externally or by in-situ polymerization.

In some aspects, a selected amount of a thermally processible and water soluble, surface treatment material can be coated onto the surfaces of the superabsorbent material to provide a desired, overall thermal stickiness of the material. Exemplary coatings and processes are described in U.S. Patent Publication No. 2006/0004336 to Zhang et al., which is hereby incorporated by reference in its entirety in a manner that is consistent herewith. The coating layer can cover the whole (or approximately the whole) outer-surface of the superabsorbent material when the material is coated by a solution coating process. The resulting morphology can produce a significantly larger amount of surface area that is coated by the thermally sticky coating material while employing a reduced amount of the coating material. As a result, the coating material can be utilized with significantly higher efficiency. The higher utilization efficiency of the coating material can increase the number of bonds formed between a superabsorbent material and other materials and/or fibers in the absorbent composite. Suitable thermally processible and water soluble polymers include, but are not limited to, maleated propylene, modified polyvinyl alcohol, polyethylene oxide, polypropylene oxide, ethylene oxide-propylene oxide copolymer, polyethylene glycol, polypropylene glycol, ethylene glycol-propylene glycol copolymer, polyacrylic acid copolymers, quaternary ammonium acrylate, methacrylate, or acrylamide copolymers, modified polysaccharides, such as hydroxypropyl cellulose, methyl cellulose, methyl ethyl cellulose, polyethylene imine, as well as mixtures or other combinations thereof.

The molecular weight of the thermal coating can be important. In general, a higher molecular weight polymer can provide a desired, higher intrinsic cohesion. When the molecular weight of a coating polymer is too high, however, an aqueous solution of the coating polymer can have an excessive level of viscosity, which may potentially create difficulties in conducting desired surface treating operations. In some aspects of the invention, the molecular weight of a thermally processible and water soluble surface treatment (e.g. coating) polymer can be at least a minimum of about 5,000. The molecular weight can alternatively be at least about 10,000, and can optionally be about 50,000. In another aspect the molecular weight of the surface treatment material can be up to a maximum of about 10,000,000. The molecular weight can alternatively be not more than about 1,000,000, and can optionally be not more than about 500,000 to provide improved benefits.

The thermally processible and water soluble surface treatment material can desirably be coated onto the surface of the superabsorbent particle by employing an aqueous solution of the surface treatment material to promote the formation of a desired Interpenetrating Polymer Network. When the surface treatment material (e.g. polymer) is dissolved into an operative aqueous solution, the solution can have a selected concentration of the surface treatment material. In a particular feature, the concentration of the surface treatment material in the solution can be at least a minimum of about 0.01% by weight. The concentration of the surface treatment material can alternatively be at least about 0.1% by weight, and can optionally be at least about 0.5% by weight to provide improved benefits. In other aspects, the concentration of the surface treatment material can be up to a maximum of about 20% by weight, or more. The concentration of the surface treatment material can alternatively be up to about 10% by weight, and can optionally be up to about 5% by weight to provide improved effectiveness.

If the molecular weight and/or concentration of the surface treatment material is outside the desired values, the treatment material may not adequately provide a desired, deeper penetration of the coated polymer into the superabsorbent polymer material. As a result, the superabsorbent material may exhibit insufficient levels of thermal stickiness, bonding strength and absorbency.

In some aspects, the formulation of the thermal coating can be adjusted so that the coating is generally inactive at ambient conditions in order to be dispensed and metered with standard superabsorbent processing methods. The coating can be thermally active and sticky at temperatures encountered in the commingling and formation zone of the coform process to facilitate attachment to the melt blown fibers.

In some aspects of the present invention, at least one of the regions of the absorbent composite may additionally or alternatively include materials such as surfactants, ion exchange resin particles, moisturizers, emollients, perfumes, fluid modifiers, odor control additives, and combinations thereof.

The absorbent components can have corresponding configurations of absorbent capacities, configurations of densities, configurations of basis weights and/or configurations of sizes which are selectively constructed and arranged to provide desired combinations of liquid intake time, absorbent saturation capacity, growth, tensile, softness, extension, shape maintenance, and aesthetics.

The absorbent composite or its selected regions can have any desirable basis weight. For example, in some aspects, the low capacity region may have a basis weight between about 5 gsm and about 100 gsm, such as between about 10 gsm and about 50 gsm. In other aspects, the high capacity region may have a basis weight between about 25 and about 1000 gsm, such as between about 300 gsm and about 800 gsm, or between about 500 to about 700 gsm.

In some aspects, the composite may exhibit improved absorbent properties, such as saturated capacity, as compared to conventional stretchable absorbent composites. In one example, the composite exhibits an absorbency of at least 16 g/g as measured by the 0.5 psi Saturated Capacity Test described below. In other aspects, the composite may exhibit improved fluid intake rates as compared to conventional stretchable absorbent composites. In particular examples, the composite exhibits an intake rate of at least 0.4 ml/sec after the first, second, and/or third fluid insult, as measured by the Fluid Intake Rate Test described below.

In some aspects, the absorbent composite can have a Geometric Mean Growth of about 20% or less, such as about 10% or less as measured by the Geometric Mean Growth Test described below. In other aspects, the absorbent composite can have an MD Modulus at least 75 times greater, such as 100 times greater, than the ZD tensile strength as measured by the MD Modulus Test and the ZD Tensile Test, respectively, each described below. In still other aspects, the absorbent composite can have a geometric mean modulus of less than 1 MPa as measured by the Geometric Mean Modulus Test described below. In still other aspects, the absorbent composite can have an average surface softness of less than 0.03 MMD at 25 grams as measured by the KES Surface Softness Test described below.

It is a feature of the present invention that the absorbent composite is not limited to merely two regions, but rather may have any number of regions, each comprising a desired set of properties. The regions can be selectively located in any desired shape and size within the absorbent composite. Typically, the high capacity region is intended primarily for absorbing fluids and is located in at least an area intended to be in close proximity to the discharge orifice of the user. The low capacity region is intended primarily for superabsorbent containment, preventing loss of superabsorbent from the high capacity region and for aesthetic purposes and is typically located in areas that will contact skin to form a fluid permeable barrier between the skin and the high capacity region.

The absorbent composite of the present invention can additionally comprise at least one “target zone,” as defined above. At least a portion of any of the regions may be located in the target zone. For example, in one aspect, at least a portion of the high capacity region is located in the target zone. The target zone may suitably comprise the entire length of an absorbent composite or may comprise a specific area as desired. For example, the target zone may comprise at least about 25% of the area of the absorbent composite, such as at least about 50% or at least about 75% of the area.

The absorbent composite of the present invention can be stretchable, and may further be elastically stretchable, at least about 30%, such as at least about 50%, or at least about 75%, based on length in an unstretched condition. Alternatively, the absorbent composite of the present invention can be extensible, and/or elastically extensible at about 200% or less, such as about 100% or less based on length in an unstretched condition to provide desired effectiveness. In particular aspects, the absorbent composite can have an MD elongation at 50% extension of at least 100 g_{f}/inch, as measured by the Elongation Test described below. In other particular aspects, the absorbent composite can have a CD elongation at 50% extension of at least 100 g_{f}/inch, as measured by the Elongation Test.

If the stretchability parameter is outside the desired values, the absorbent composite may not sufficiently elongate and/or retract to provide desired levels of fit and conformance to the shape of the user. A donning of a product that includes such an absorbent composite would then be more difficult. For example, training pant products may be accidentally extended to large amounts before use, and the absorbent system may rip and tear. As a result, the absorbent composite may exhibit excessive sag, droop and leakage problems.

In some aspects, superabsorbent material and or cellulosic fiber can be combined with the elastomeric polymer during formation of the absorbent composite in a meltblowing operation to form coform materials. Where the absorbent composite includes additional materials, those materials can also be mixed with the superabsorbent material and/or cellulosic fibers, and the mixture can then be operatively combined with the meltblown polymer fibers.

The absorbent composite can be formed using methods known in the art. While not being limited to the specific method of manufacture, the absorbent composite can utilize a meltblown process and can further be formed on a coform line. Exemplary meltblown processes are described in various patents and publications, including NRL Report 4364, “Manufacture of Super-Fine Organic Fibers” by V. A. Wendt, E. L. Boone and C. D. Fluharty; NRL Report 5265, “An Improved Device For the Formation of Super-Fine Thermoplastic Fibers” by K. D. Lawrence, R. T. Lukas and J. A. Young; and U.S. Pat. Nos. 3,849,241 to Butin et al. and 5,350,624 to Georger et al., all of which are hereby incorporated by reference in a manner that is consistent herewith. Suitable techniques and systems for producing nonwoven fibrous webs which include meltblown fibers are also disclosed in U.S. Pat. Nos. 4,100,324 to Anderson et al., 5,350,624 to Georger et al. and 5,508,102 to Georger et al., all of which are incorporated herein by reference in a manner that is consistent herewith.

To form “coform” materials, additional materials are mixed with the meltblown fibers as the fibers are deposited onto a forming surface. Such components include, but are not limited to, superabsorbent materials and fluff, such as wood pulp fibers, which may be injected into a meltblown fiber stream so as to be entrapped and/or bonded to the meltblown fibers. Exemplary coform processes are described in U.S. Pat. Nos. 4,100,324 to Anderson et al.; 4,587,154 to Hotchkiss et al.; 4,604,313 to McFarland et al.; 4,655,757 to McFarland et al.; 4,724,114 to McFarland et al.; 4,100,324 to Anderson et al.; and U.K. Patent GB 2,151,272 to Minto et al., each of which is incorporated herein by reference in a manner that is consistent herewith. Absorbent elastomeric meltblown webs containing high amounts of superabsorbent are described in U.S. Pat. No. 6,362,389 to McDowall et al., and absorbent elastomeric meltblown webs containing high amounts of superabsorbent and low superabsorbent shakeout values are described in pending U.S. Patent Publication US2006/0004336 to X. Zhang et al., each of which is incorporated herein by reference in a manner that is consistent herewith.

One example of a method of forming at least one region of the absorbent composite of the present invention is illustrated in FIG. 4. The dimensions of the apparatus in FIG. 4 are described herein by way of example. Other types of apparatus having different dimensions and/or different structures may also be used to form the absorbent composite. As shown in FIG. 4, elastomeric material 72 in the form of pellets can be fed through two pellet hoppers 74 into two single screw extruders 76 that each feed a spin pump 78. The elastomeric material 72 may be a multicomponent elastomer blend available under the trade designation VISTMAXX VM 2370 from ExxonMobil Chemical Company (a business having offices located in Houston, Tex. U.S.A.), as well as others mentioned herein. Each spin pump 78 feeds the elastomeric material 72 to a separate meltblown die 80. Each meltblown die 80 may have 30 holes per inch (hpi). The die angle may be adjusted anywhere between 0 and 70 degrees from horizontal, and is suitably set at about 28 degrees. The forming height may be at a maximum of about 20 inches (51 cm), but this restriction may differ with different equipment.

A chute 82 having a width of about 24 inches wide may be positioned between the meltblown dies 80. The depth, or thickness, of the chute 82 may be adjustable in a range from about 0.5 to about 1.25 inches (1.27-3.18 cm), or from about 0.75 to about 1.0 inch (1.91-2.54 cm). A picker 144 connects to the top of the chute 82. The picker 144 is used to fiberize the pulp (fluff) fibers 86. The picker 144 may be limited to processing low strength or debonded (treated) pulps, in which case the picker 144 may limit the illustrated method to a very small range of pulp types. In contrast to conventional hammermills that use hammers to impact the pulp fibers repeatedly, the picker 144 uses small teeth to tear the pulp fibers 86 apart. Suitable pulp fibers 86 for use in the method illustrated in FIG. 4 include those mentioned herein, such as CF405 (available from Weyerhaeuser Co., a business having offices located in Federal Way, Wash. U.S.A.).

At an end of the chute 82 opposite the picker 144 is a superabsorbent material feeder 88. The feeder 88 pours the superabsorbent material 90 of the present invention into a hole 92 in a pipe 94 which then feeds into a blower fan 96. Past the blower fan 96 is a length of 4-inch (10.2 cm) diameter pipe 98 sufficient for developing a fully developed turbulent flow at about 5,000 feet per minute, which allows the superabsorbent material 90 to become distributed. The pipe 98 widens from a 4-inch (10.2 cm) diameter to the 24-inch by 0.75-inch (1.91 cm) chute 82, at which point the superabsorbent material 90 mixes with the pulp fibers 86 and the mixture falls straight down and gets mixed on either side at an approximately 45-degree angle with the elastomeric material 72. The mixture of superabsorbent material 90, pulp fibers 86, and elastomeric material 72 falls onto a wire conveyor 100 moving from about 14 to about 35 feet per minute. However, before hitting the wire conveyor 100, a spray boom 102 optionally sprays an aqueous surfactant mixture 104 in a mist through the mixture, thereby rendering the resulting absorbent composite region 45 wettable. The surfactant mixture 104 may be a 1:3 mixture of GLUCOPON 220 UP (available from Cognis Corporation having a place of business in Cincinnati, Ohio, U.S.A.) and AHCOVEL Base N-62 (available from Uniqema, having a place of business in New Castle, Del., U.S.A.). An under-wire vacuum 106 is positioned beneath the conveyor 100 to assist in forming the absorbent composite region 45.

To form an absorbent composite having multiple regions that are stratified in accordance with the present invention, a first region may be formed using an apparatus such as that described above. The material can be wound into a roll and positioned on a unwind. The material can then be threaded through the machine and another region having desired properties can be applied. This process can be repeated for as many regions as are desired. Alternatively, additional sets of dies having structures such as described above could be placed in series such that an absorbent composite having multiple stratified regions could be produced in a more continuous process. For example, a first set can deposit a low capacity region, a second set can deposit a high capacity region onto the low capacity region, and an optional third set can deposit an additional region onto the high capacity region.

In particular configurations, selected processing parameters can be appropriately controlled to produce desired characteristics in the absorbent composite of the present invention. For example:

Melt-temperature—Higher melt-temperatures can provide better containment of the incorporated superabsorbent material (i.e., less shake-out), as well as better fiber intermingling between the stratified regions. The higher polymer temperatures will result in increased polymer fiber surface area, prolonged duration of tackiness within the commingled zone, and increased activation of the optional thermal coating on the superabsorbent.

Air Gap—The air gap spacing is measured from a knife edge of the meltblowing die tip to an inside edge of the air plate in the meltblowing die. In a typical arrangement, the air gap spacing can be within the range of about 0.015-0.084 inches (0.038-0.213 cm). In one particular example, the air gap was 0.045 inches (0.114 cm). A higher, primary-air velocity can create smaller fibers and help provide a higher containment of the material. The smaller fibers can provide an increased amount of surface area.

Die angle—The die angle is the pitch of the die assemblies in relationship to the horizontal. In a two die system, the dies are facing each other and usually pitched downwards to intersect with the material stream exiting the nozzle. A large angle from the horizontal can reduce contact between the polymer fibers and the material, resulting in poor commingling of the materials. The resulting non-homogenous absorbent can have deficiencies such as poor superabsorbent containment as well as a polymeric “skin” on either or both sides which will limit intake. The die angle can, for example, be within the range of about 0 to 65 degrees from horizontal. In one example, the die angle was 33 degrees from horizontal. In another example, the die angle was 57 degrees from horizontal. In yet another example, the die angle was 65 degrees from horizontal.

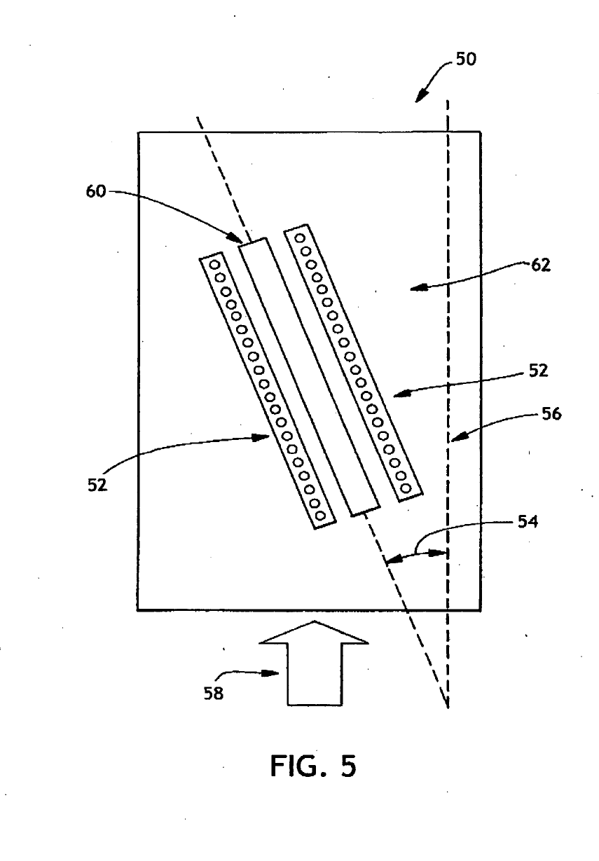

Die Orientation—The die orientation is the angle of the die and nozzle assembly in relation to the machine centerline. In many coform applications for the production of roll goods, the die assembly is perpendicular to the running direction of the machine. However, in the production of a relatively high basis weight of a personal care absorbent, throughput limitations require that the meltblown assembly be angled in order to achieve the desired throughput levels required. As seen in FIG. 5, a top view perspective of an example coform process 50 is shown. Shown is a forming fabric 62 which travels in the machine-direction 58 and a set of dies 52 that are oriented at an angle 54 from the machine-direction centerline 56 based on the machine-direction 58. The die orientation can, for example, be within the range of about 0 to 90 degrees, such within about 2 to 45 degrees, from the machine-direction centerline. In one example, the die orientation was 18 degrees from the machine-direction centerline. In another example, the die orientation was 22 degrees from the machine-direction centerline. The coform process 50 may also have a pulp chute 60 present. The pulp chute 60 may also be oriented at an angle 54, which would typically be the same as the die orientation 54.

By incorporating its various features and configurations, alone and in operative combinations, the invention can provide an improved absorbent composite having a desired combination of stretchability, absorbent capacity, and dimensional stability. The absorbent composite can be manufactured to provide selected regions of absorbent component and/or elastomeric polymer quantities. An absorbent article comprising the absorbent composite of the present invention can be less susceptible to premature leakage, and can provide improved comfort and fit, improved protection and increased confidence to the wearer. For example, the absorbent composite of the present invention, when in an absorbent article, can help eliminate bunching, discomfort, and worry by improving the dimensional stability of the absorbent composite in the absorbent article. Additional benefits can be obtained if the absorbent composite of the present invention is incorporated into a stretchable absorbent article.

The present invention is primarily described herein in combination with an absorbent disposable training pant. However, it is readily apparent to one skilled in the art, based on the disclosure herein, that the absorbent composite described herein can also be used in combination with numerous other disposable absorbent articles including, but not limited to, other personal care absorbent articles, health/medical absorbent articles, household/industrial absorbent articles, sports accessory absorbent articles, and the like without departing from the scope of the present invention.

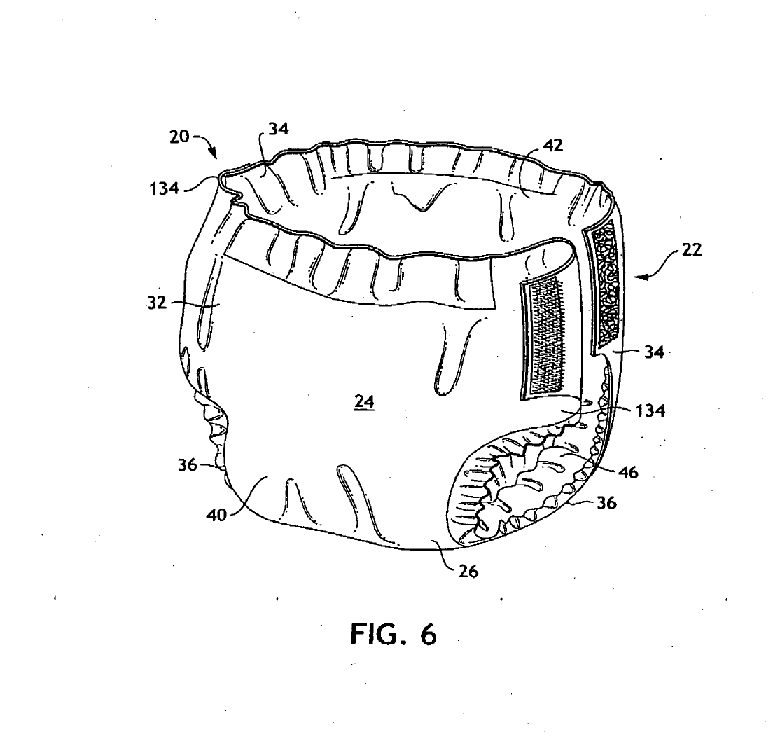

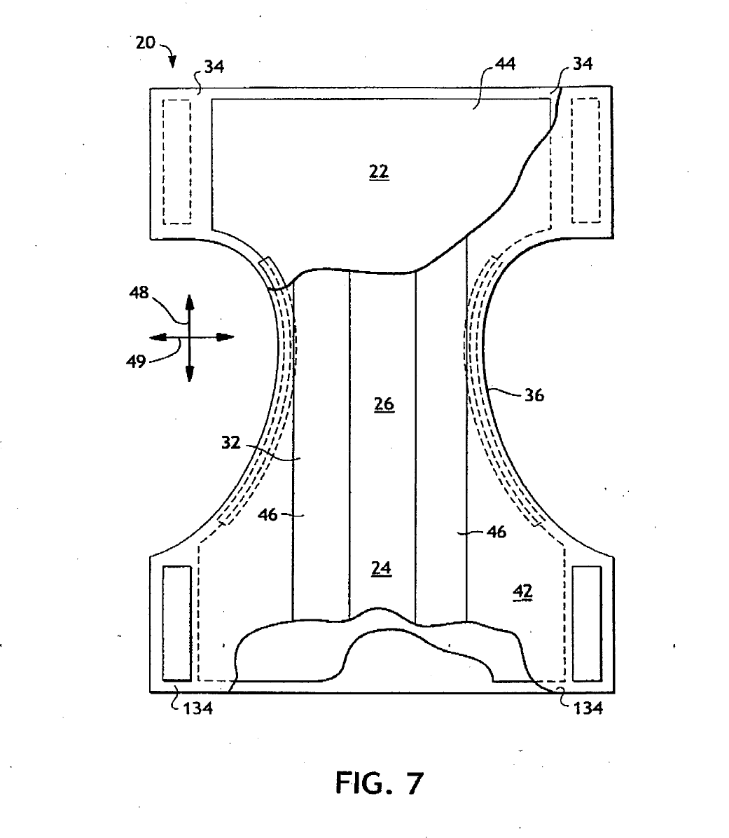

Referring to FIGS. 6 and 7 for exemplary purposes, a training pant which may incorporate the present invention is shown. Various materials and methods for constructing training pants are disclosed in PCT Patent Application WO 00/37009 published Jun. 29, 2000 by A. Fletcher et al.; U.S. Pat. Nos. 4,940,464 to Van Gompel et al.; 5,766,389 to Brandon et al., and 6,645,190 to Olson et al., all of which are incorporated herein by reference in a manner that is consistent herewith.

FIG. 6 illustrates a training pant in a partially fastened condition, and FIG. 7 illustrates a training pant in an opened and unfolded state. The training pant defines a longitudinal direction 48 that extends from the front of the training pant when worn to the back of the training pant. Perpendicular to the longitudinal direction 48 is a lateral direction 49.

The pair of training pants defines a front region 22, a back region 24, and a crotch region 26 extending longitudinally between and interconnecting the front and back regions. The pant also defines an inner surface adapted in use (e.g., positioned relative to the other components of the pant) to be disposed toward the wearer, and an outer surface opposite the inner surface. The training pant has a pair of laterally opposite side edges and a pair of longitudinally opposite waist edges.

The illustrated pant 20 may include a chassis 32, a pair of laterally opposite front side panels 34 extending laterally outward at the front region 22 and a pair of laterally opposite back side panels 134 extending laterally outward at the back region 24.

The chassis 32 includes a backsheet 40 and a topsheet 42 that may be joined to the backsheet 40 in a superimposed relation therewith by adhesives, ultrasonic bonds, thermal bonds or other conventional techniques. The chassis 32 further includes the absorbent composite 44 of the present invention such as shown in FIG. 7 disposed between the backsheet 40 and the topsheet 42 for absorbing fluid body exudates exuded by the wearer, and may further include a pair of containment flaps 46 secured to the topsheet 42 or the absorbent composite 44 for inhibiting the lateral flow of body exudates.

The backsheet 40, the topsheet 42 and the absorbent composite 44 may be made from many different materials known to those skilled in the art. All three layers, for instance, may be extensible and/or elastically extensible. Further, the stretch properties of each layer may vary in order to control the overall stretch properties of the product.

The backsheet 40, for instance, may be breathable and/or may be fluid impermeable. The backsheet 40 may be constructed of a single layer, multiple layers, laminates, spunbond fabrics, films, meltblown fabrics, elastic netting, microporous webs or bonded-carded-webs. The backsheet 40, for instance, can be a single layer of a fluid impermeable material, or alternatively can be a multi-layered laminate structure in which at least one of the layers is fluid impermeable.

The backsheet 40 can be biaxially extensible and optionally biaxially elastic. Elastic non-woven laminate webs that can be used as the backsheet 40 include a non-woven material joined to one or more gatherable non-woven webs or films. Stretch Bonded Laminates (SBL) and Neck Bonded Laminates (NBL) are examples of elastomeric composites.

Examples of suitable nonwoven materials are spunbond-meltblown fabrics, spunbond-meltblown-spunbond fabrics, spunbond fabrics, or laminates of such fabrics with films, or other nonwoven webs. Elastomeric materials may include cast or blown films, meltblown fabrics or spunbond fabrics composed of polyethylene, polypropylene, or polyolefin elastomers, as well as combinations thereof. The elastomeric materials may include PEBAX elastomer (available from AtoFina Chemicals, Inc., a business having offices located in Philadelphia, Pa., U.S.A.), HYTREL elastomeric polyester (available from Invista, a business having offices located in Wichita, Kans., U.S.A.), KRATON elastomer (available from Kraton Polymers, a business having offices located in Houston, Tex., U.S.A.), or strands of LYCRA elastomer (available from Invista), or the like, as well as combinations thereof. The backsheet 40 may include materials that have elastomeric properties through a mechanical process, printing process, heating process or chemical treatment. For example, such materials may be apertured, creped, neck-stretched, heat activated, embossed, and micro-strained, and may be in the form of films, webs, and laminates.

One example of a suitable material for a biaxially stretchable backsheet 40 is a breathable elastic film/nonwoven laminate, such as described in U.S. Pat. No. 5,883,028, to Morman et al., incorporated herein by reference in a manner that is consistent herewith. Examples of materials having two-way stretchability and retractability are disclosed in U.S. Pat. Nos. 5,116,662 to Morman and 5,114,781 to Morman, each of which is incorporated herein by reference in a manner that is consistent herewith. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

In some aspects, one or more regions of the invention may be designed to have a function similar to the backsheet, which allows for the elimination of a separate backsheet layer.

The topsheet 42 is suitably compliant, soft-feeling and non-irritating to the wearer's skin. The topsheet 42 is also sufficiently liquid permeable to permit liquid body exudates to readily penetrate through its thickness to the absorbent composite 44. A suitable topsheet 42 may be manufactured from a wide selection of web materials, such as porous foams, reticulated foams, apertured plastic films, woven and non-woven webs, or a combination of any such materials. For example, the topsheet 42 may include a meltblown web, a spunbonded web, or a bonded-carded-web composed of natural fibers, synthetic fibers or combinations thereof. The topsheet 42 may be composed of a substantially hydrophobic material, and the hydrophobic material may optionally be treated with a surfactant or otherwise processed to impart a desired level of wettability and hydrophilicity.

The topsheet 42 may also be extensible and/or elastically extensible. Suitable elastomeric materials for construction of the topsheet 42 can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from Noveon, a business having offices located in Cleveland, Ohio, U.S.A.), or PEBAX elastomers. The topsheet 42 can also be made from extensible materials such as those described in U.S. Pat. No. 6,552,245 to Roessler et al. which is incorporated herein by reference in a manner that is consistent herewith. The topsheet 42 can also be made from biaxially stretchable materials as described in U.S. Pat. No. 6,641,134 filed to Vukos et al. which is incorporated herein by reference in a manner that is consistent herewith.

In some aspects, one or more regions of the invention may be designed to have a function similar to the topsheet, which allows for the elimination of a separate topsheet layer.

The article 20 can further comprise the absorbent composite 44 of the present invention. The absorbent composite 44 may have any of a number of shapes. For example, it may have a 2-dimensional or 3-dimensional configuration, and may be rectangular shaped, triangular shaped, oval shaped, race-track shaped, I-shaped, generally hourglass shaped, T-shaped and the like. It is often suitable for the absorbent composite 44 to be narrower in the crotch portion 26 than in the rear 24 or front 22 portion(s). The absorbent composite 44 can be attached in an absorbent article, such as to the backsheet 40 and/or the topsheet 42 for example, by bonding means known in the art, such as ultrasonic, pressure, adhesive, aperturing, heat, sewing thread or strand, autogenous or self-adhering, hook-and-loop, or any combination thereof.

The article 20 can optionally further include a surge management layer (not shown) which may be located adjacent the absorbent composite 44 and attached to various components in the article 20 such as the absorbent composite 44 or the topsheet 42 by methods known in the art, such as by using an adhesive. In general, a surge management layer helps to quickly acquire and diffuse surges or gushes of liquid that may be rapidly introduced into the absorbent structure of the article. The surge management layer can temporarily store the liquid prior to releasing it into the storage or retention portions of the absorbent composite 44. Examples of suitable surge management layers are described in U.S. Pat. Nos. 5,486,166 to Bishop et al.; 5,490,846 to Ellis et al.; and 5,820,973 to Dodge et al., each of which is incorporated herein by reference in a manner that is consistent herewith.

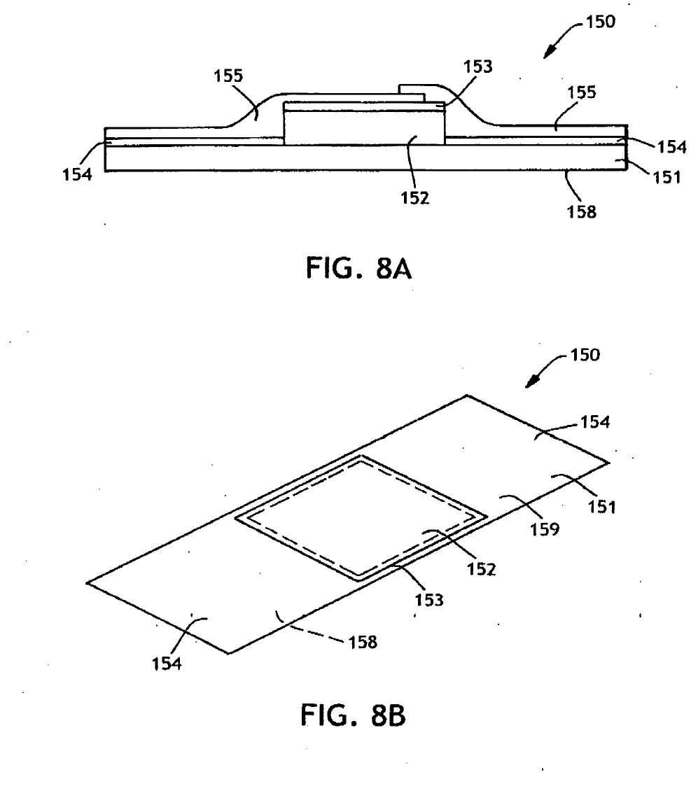

In addition to the absorbent articles described above, the absorbent composite of the present invention may be used as an absorbent bandage. Attention is directed to FIGS. 8A and 8B, which show a possible configuration for a bandage of the present invention. FIG. 8A shows a cross-section view of the absorbent bandage with optional layers described below. FIG. 8B shows a perspective view of the bandage of the present invention with some of the optional or removable layers not being shown. The absorbent bandage 150 has a strip 151 of material having a body-facing side 159 and a second side 158 which is opposite the body-facing side. The strip is essentially a backsheet and is desirably prepared from the same materials described above for the backsheet. In addition, the strip may be an apertured material, such as an apertured film, or material which is otherwise gas permeable, such as a gas permeable film. The strip 151 supports the absorbent composite 152 of the present invention which is attached to the body facing side 159 of the strip. In addition, an optional absorbent protective layer 153 may be applied to the absorbent composite 152 and can be coextensive with the strip 151.

The absorbent bandage 150 of the present invention may also have a pressure sensitive adhesive 154 applied to the body-facing side 159 of the strip 151. Any pressure sensitive adhesive may be used, provided that the pressure sensitive adhesive does not irritate the skin of the user. Suitably, the pressure sensitive adhesive is a conventional pressure sensitive adhesive which is currently used on similar conventional bandages. This pressure sensitive adhesive is preferably not placed on the absorbent composite 152 or on the absorbent protective layer 153 in the area of the absorbent composite 52. If the absorbent protective layer is coextensive with the strip 151, then the adhesive may be applied to areas of the absorbent protective layer 153 where the absorbent composite 152 is not located. By having the pressure sensitive adhesive on the strip 151, the bandage is allowed to be secured to the skin of a user in need of the bandage. To protect the pressure sensitive adhesive and the absorbent, a release strip 155 can be placed on the body facing side 159 of the bandage. The release liner may be removably secured to the article attachment adhesive and serves to prevent premature contamination of the adhesive before the absorbent article is secured to, for example, the skin. The release liner may be placed on the body facing side of the bandage in a single piece (not shown) or in multiple pieces, as is shown in FIG. 8A.

In another aspect of the present invention, the absorbent composite of the bandage may be placed between a folded strip. If this method is used to form the bandage, the strip is suitably fluid permeable.

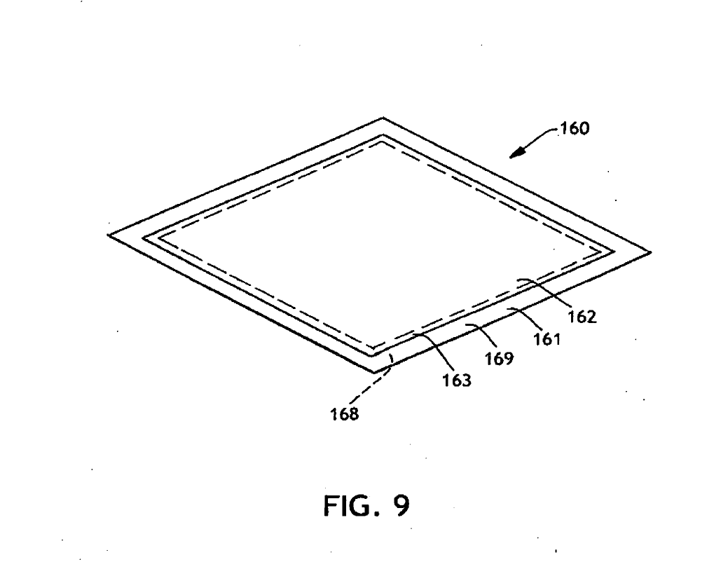

Absorbent furniture and/or bed pads or liners are also included within the present invention. As is shown in FIG. 9, a furniture or bed pad or liner 160 (hereinafter referred to as a “pad”) is shown in perspective. The pad 160 has a fluid impermeable backsheet 161 having a furniture-facing side or surface 168 and an upward facing side or surface 169 which is opposite the furniture-facing side or surface 168. The fluid impermeable backsheet 161 supports the absorbent composite 162 of the present invention which is attached to the upward facing side 169 of the fluid impermeable backsheet. In addition, an optional absorbent protective layer 163 may be applied to the absorbent composite. The optional substrate layer of the absorbent composite can be the fluid impermeable layer 161 or the absorbent protective layer 163 of the pad.

To hold the pad in place, the furniture-facing side 168 of the pad may contain a pressure sensitive adhesive, a high friction coating or other suitable material which will aid in keeping the pad in place during use. The pad of the present invention can be used in a wide variety of applications including placement on chairs, sofas, beds, car seats and the like to absorb any fluid which may come into contact with the pad.

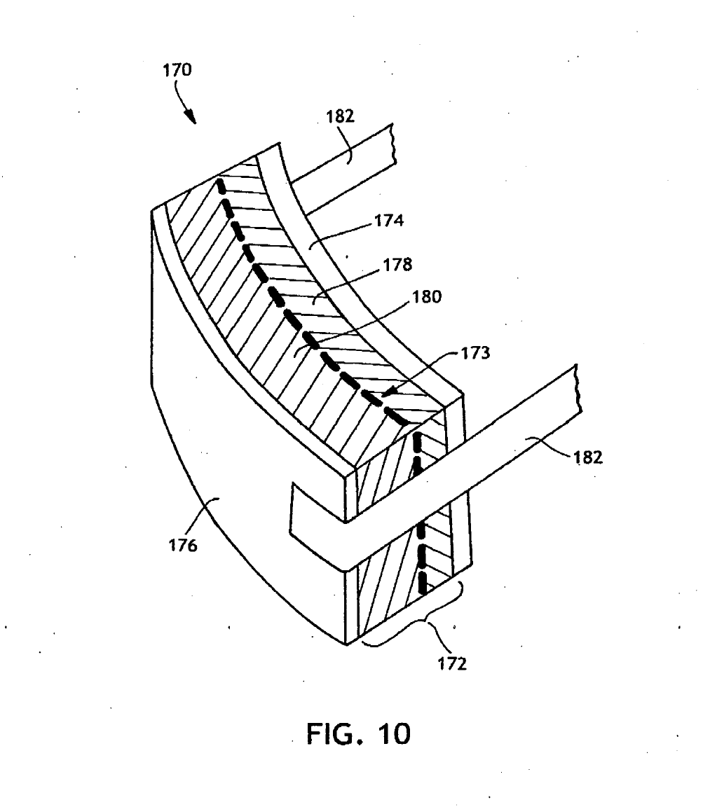

Sports or construction accessories, such as an absorbent headband for absorbing perspiration or drying off equipment are also included within the present invention. As is shown in FIG. 10, a highly absorbent sweatband 170 is shown in perspective. The sweatband 170 has an absorbent composite 172 disposed between an optional topsheet 174 and/or an optional fluid impervious backsheet 176. The absorbent composite 172 has a low capacity region 178 and a high capacity region 180, and could include an optional additional region (not shown) if desired. The regions are stratified through polymeric bonding and polymer fiber intermingling, as shown by broken line 173. The sweatband can be useful where dimensional stability is needed to maintain good contact with the skin to intercept perspiration prior to contact with the hands or eyes. The elastomeric nature of the article 170 allows the band to be fitted on the user's head or wrist while the nature of the invention retains exceptional dimensional stability to ensure contact with the skin. The low capacity region 178 can be positioned towards the user's skin and can maintain a comfortable feel to the user. Velcro or other fastening device 182 can be used to facilitate adjustment or comfort.

The present invention may be better understood with reference to the following examples.

### EXAMPLES

The following examples are provided to further illustrate the present invention and do not limit the scope of the claims. Unless otherwise stated, all parts and percentages are by weight. The examples were made on a meltblown coform process, similar to those described in U.S. Pat. Nos. 4,100,324 to Anderson et al. and 6,362,389 to McDowall et al., previously incorporated herein by reference. Unless otherwise stated, process conditions for each example using the meltblown coform process were as seen in Table 1 below. All absorbent composites were formed on a CEREX carrier sheet, Product #23050, a nonwoven made of spunbond nylon having a slit width of both 8 and 10 inches, and a basis weight of 0.5 osy (available from Cerex Advanced Fabrics, Inc., having a place of business in Cantonment, Fla., U.S.A.) which was subsequently removed for testing.

TABLE 1
Key Process Conditions for Coform Examples

| | EXAMPLES | EXAMPLES | EXAMPLES |
|---|---|---|---|
| | 1-3 | 4-6 | 7-16 |
| Line Speed, DPM | 150 | 200 | 200 |
| (FPM = DPM × | | | |
| 1.125) | | | |
| Die Orientation | 22 | 22 | 22 |
| (degrees from | | | (unless |
| machine direction | | | otherwise |
| centerline) | | | noted) |
| Die Angle (degrees | 57 | 57 | 57 |
| from horizontal) | | | |
| Air Gap (cm per side) | 0.114 | 0.114 | 0.114 |
| Tip Configuration | Stick-out | Stick-out | Stick-out |
| Air Plate | non-restricted | non-restricted | non-restricted |
| Die Tip-To-Tip | 14.0 | 14.0 | 14.0 |
| Distance (cm) | | | |
| Forming Height (cm) | 35.6 | 35.6 | 35.6 |
| Polymer | VISTAMAXX | VISTAMAXX | VISTAMAXX |
| | VM 2370 | VM 2370 | VM 2370 |
| SAM | SR1642 | SR1642 | SR1642 |
| Fluff (cellulosic fiber) | CF405 | CF405 | CF405 |
| | (debonded) | (debonded) | (debonded) |
| Surfactant | None | 1.5% | 1.5% |
| Primary Air Pressure | 2.75 | 2.5 | 2.0 |
| (psi) | | | |
| Primary Air Flow | 9.7 | 9.0 | 7.7 |
| (SCFM/in) | | | |
| Polymer Melt | 243 | 243 | 216 |
| Temperature, ° C. | | | |
| Carrier | Yes | Yes | Yes |
| | (Cerex) | (Cerex) | (Cerex) |

### Comparative Example 1

Comparative Example 1 was a homogeneous absorbent composite (i.e., a single region) made to a basis weight of 500-gsm and is designated the “control” for most comparisons in this specification. This sample represents a conventional stretchable absorbent composite. The overall composition was 75% superabsorbent, 10% fluff, and 15% meltblown polymer.

This sample exhibited an undesirable amount of wet in-plane growth when tested.

### Comparative Example 2

Comparative Example 2 was a homogeneous absorbent composite made to a basis weight of 530-gsm and a superabsorbent basis weight of 375-gsm. This sample represents a conventional stretchable absorbent composite. The overall composition was 70.8% superabsorbent, 15% fluff, and 14.2% meltblown polymer.

This sample exhibited lower wet in-plane growth than Sample 1 when tested but at levels that were still undesirable.

### Comparative Example 3

Comparative Example 3 was a homogeneous absorbent composite made to a basis weight of 562-gsm and a superabsorbent basis weight of 375-gsm. This sample represents a conventional stretchable absorbent composite. The overall composition was 66.7% superabsorbent, 20% fluff, and 13.3% meltblown polymer.

This sample exhibited lower wet in-plane growth than Sample 1 when tested but at levels that were still undesirable.

### Example 4

Example 4 was an absorbent composite produced with a top low absorbent capacity region, a middle highly absorbent capacity region, and a bottom low absorbent capacity region made to a composite basis weight of 500-gsm. The overall composition was similar to Comparative Example 1 with 75% superabsorbent, 10% fluff, and 15% meltblown polymer. The meltblown polymer was distributed equally in all regions with a basis weight of 25-gsm per region. The middle region composition was 5.8% melt blown polymer, 7.0% fluff and 87.2% SAM and a total region basis weight of 430-gsm.

The two outer region compositions were each 10-gsm fluff and 25-gsm meltblown polymer and each outer region was made to a basis weight of 40-gsm and a 70:30 MB:Fluff ratio. No wetting agent was used.

The Geometric Mean Growth for this example was found to be less than 20% (i.e., 16.1%), as measured by the Geometric Mean Growth Test.

### Example 5

Example 5 was an absorbent composite produced with a top low capacity region, a middle high capacity region, and a bottom low capacity region made to a composite basis weight of 490-gsm. The overall meltblown content for this code was 13.3%.

Each outer region composition was 10-gsm fluff and 25-gsm meltblown polymer resulting in a basis weight of 40-gsm and a 30:70 MB:Fluff ratio. The polymer content in the high capacity middle region was 3.6%. The overall meltblown content for this Example was 13.3% versus 15% for Comparative Example 1 and the stratified Example 4. No wetting agent was used.

The Geometric Mean Growth for this example was found to be less than 15% (i.e., 12.3%), as measured by the Geometric Mean Growth Test.

### Example 6

Example 6 was an absorbent composite produced with a top low capacity region, a middle high capacity region, and a bottom low capacity region made to a composite basis weight of 480-gsm. The overall meltblown content for this Example was 9.4%.

Each outer region composition was 15-gsm fluff and 15-gsm meltblown polymer and each outer region was made to a basis weight of 30-gsm and a 50:50 MB:Fluff ratio. The middle region composition was 3.6% meltblown polymer, 7.1% fluff and 89.3% SAM and a total region basis weight of 420-gsm.

The Geometric Mean Growth for this example was found to be less than 10% (i.e., 8.9%), as measured by the Geometric Mean Growth Test.

### Example 7

Example 7 was an absorbent composite produced with a top low capacity region, a middle high capacity region, and a bottom low capacity region made to a composite basis weight of 658-gsm. The overall meltblown content for this Example was 7.6%. Each outer region composition was 50% melt blown polymer and 50% fluff and each outer region was made to a basis weight of 30-gsm. The middle region composition was 3.3% meltblown polymer, 13.3% fluff and 83.3% SAM and made to a basis weight of 598-gsm. An internal surfactant, IRGASURF HL-560 at 1.5% actives was added to the base polymer. The composites of this Example exhibited improved fast wetting (vs. non-surfactant treated materials) and maintained intake functionality over repeated insults.

### Example 8

Example 8 was an absorbent composite produced with a top low capacity region, a middle high capacity region, and a bottom low capacity region made to a composite basis weight of 648-gsm. The overall meltblown content for this Example was 9.3%. Each outer region composition was 50% meltblown polymer and 50% fluff and each outer region was made to a basis weight of 40-gsm. The middle region composition was 3.5% meltblown polymer, 14.0% fluff and 82.5% SAM and made to a basis weight of 568-gsm. IRGASURF HL-560 internal surfactant at 1.5% actives was added to the base polymer.

For this Example, the polymer melt temperature was set to 216° C. and primary air flow was set to 120 SCFM per die. The MB fiber diameter averaged approximately 16 microns.

The Geometric Mean Growth for this example was found to be less than 10% (i.e., 7.1%), as measured by the Geometric Mean Growth Test.

### Example 9

Example 9 was an absorbent composite produced with a top low capacity region, a middle high capacity region, and a bottom low capacity region made to a composite basis weight of 648-gsm. The overall meltblown content for this code was 8.0%. Each outer region composition was 40% meltblown polymer and 60% fluff and each outer region was made to a basis weight of 40-gsm. The middle region composition was 3.5% meltblown polymer, 14.0% fluff and 82.5% SAM and made to a basis weight of 568-gsm. Process conditions were the same as those used to produce Example 8. IRGASURF HL-560 internal surfactant at 1.5% actives was added to the base polymer.

### Example 10

Example 10 was an absorbent composite produced with a top low capacity region, a middle high capacity region, and a bottom low capacity region made to a composite basis weight of 642-gsm. The overall meltblown content for this code was 8.4%. Each outer region composition was 50% meltblown polymer and 50% fluff and each outer region was made to a basis weight of 40-gsm. The middle region composition was 2.5% meltblown polymer, 14.2% fluff and 83.3% SAM and made to a basis weight of 562-gsm. Process conditions were the same as those used to produce Example 8. IRGASURF HL-560 internal surfactant at 1.5% actives was added to the base polymer.

### Example 11

Example 11 was an absorbent composite produced with a top low capacity region, a middle high capacity region, and a bottom low capacity region made to a composite basis weight of 598-gsm. The overall meltblown content for this code was 8.4%. Each outer region composition was 100% meltblown polymer and 0% fluff and each outer region was made to a basis weight of 15-gsm. The middle region composition was 3.5% melt blown polymer, 14.0% fluff and 82.5% SAM and made to a basis weight of 568-gsm. Process conditions were the same as those used to produce Example 8. IRGASURF HL-560 internal surfactant at 1.5% actives was added to the base polymer.

### Example 12

Example 12 was an absorbent composite produced with a top low capacity region, a middle high capacity region, and a bottom low capacity region made to a composite basis weight of 618-gsm. The overall meltblown content for this Example was 11.3%. Each outer region composition was 100% meltblown polymer and 0% fluff and each outer region was made to a basis weight of 25-gsm. The middle region composition was 3.5% meltblown polymer, 14.0% fluff and 82.5% SAM and made to a basis weight of 568-gsm. Process conditions were the same as those used to produce Example 8. IRGASURF HL-560 internal surfactant at 1.5% actives was added to the base polymer.

The outer region of Sample 12 was made separately and used in gel barrier testing.

### Examples 13 and 14

Examples 13 and 14 are absorbent composites having a perimeter area and a central area. These Examples were used to demonstrate how opposing outer regions could be used to create a sealed edge for improved gel containment. Each was produced by making a first outer region at a 22 degree die orientation angle (relative to the MD centerline) during the first pass. This produced a web at a normal width. Next, the die orientation angle was decreased to 18 degrees for the second pass to produce a narrower middle high capacity region. For the third pass, the die orientation was returned back to 22 degrees and the opposing outer region was applied. In this configuration, both outer regions extended beyond the region of the middle region resulting in sufficient adhesion between the top and bottom outer region portions to form a seal around the high capacity region.

Example 13 was an absorbent composite produced with a top low capacity region, a middle high capacity region, and a bottom low capacity region made to a composite basis weight of 648-gsm. The overall meltblown content for this Example was 11.3%. The outer region composition was 50% meltblown polymer and 50% fluff and each outer region was made to a basis weight of 40-gsm. The middle region composition was 3.5% meltblown polymer, 14.0% fluff and 82.5% SAM and made to a basis weight of 568-gsm. Except for the meltblown die orientation angle, process conditions were the same as those used to produce Example 8. IRGASURF HL-560 internal surfactant at an addition rate of 1.5% was added to the base polymer.

Example 14 was an absorbent composite produced with a top low capacity layer, a middle high capacity layer, and a bottom low capacity layer made to a composite basis weight of 648-gsm. The overall meltblown content for this code was 11.3%. Each outer layer composition was 100% meltblown polymer and 0% fluff and each outer layer was made to a basis weight of 15-gsm. The middle layer composition was 3.5% meltblown polymer, 14.0% fluff and 82.5% SAM and made to a basis weight of 568-gsm. Except for the meltblown die orientation angle, process conditions were the same as those used to produce Example 8. IRGASURF HL-560 internal surfactant at 1.5% actives was added to the base polymer.

### Example 15

Example 15 was an absorbent similar to the low capacity region of Example 8. The composition was 50% meltblown polymer and 50% fluff and each outer layer was made to a basis weight of 40-gsm. Example 13 consisted of this one region only.

IRGASURF HL-560 internal surfactant at an addition rate of 1.5% was added to the base polymer.

The Geometric Mean Growth for this example was found to be 0%, as measured by the Geometric Mean Growth Test.

### Example 16

Example 16 was an absorbent similar to the high capacity region of Example 8. The composition was 3.5% meltblown polymer, 14.0% fluff and 82.5% SAM and it was made to a basis weight of 568-gsm. Example 14 consisted of one region only.

IRGASURF HL-560 internal surfactant at an addition rate of 1.5% was added to the base polymer.

The Geometric Mean Growth for this example was found to be 0%, as measured by the Geometric Mean Growth Test.

Comparison of Examples 8, 15 and 16 illustrate how the properties of the individual high and low absorbent regions significantly differ compared to the properties when combined together into the claimed invention.

The examples above were tested for various properties, the results of which are summarized below in Table 2 and Table 3.

TABLE 2

| | | | | | | | | | Geometric |
|---|---|---|---|---|---|---|---|---|---|
| | Total | | | | | | % CD | % MD | Mean |
| Example | Basis Wt | | | % | Density | Sat. Cap. | GROWTH | GROWTH | Growth |
| # | (gsm) | % SAM | % Fluff | Polymer | (g/cc) | (g/g) | (%) | (%) | (%) |
| 1 | 500 | 75.0 | 10.0 | 15.0 | 0.277 | 16.5 | 33.3 | 39.3 | 36.2 |
| 2 | 530 | 70.8 | 15.0 | 14.2 | 0.245 | 18.1 | 16.7 | 30.6 | 22.6 |
| 3 | 562 | 66.7 | 20.0 | 13.3 | 0.213 | 18.0 | 16.7 | 24.0 | 20.0 |
| 4 | 500 | 75.0 | 10.0 | 15.0 | 0.281 | 17.3 | 14.4 | 17.9 | 16.1 |
| 5 | 490 | 76.5 | 10.2 | 13.3 | 0.310 | 16.7 | 10.0 | 15.2 | 12.3 |
| 6 | 480 | 78.1 | 12.5 | 9.4 | 0.276 | 19.8 | 6.7 | 11.8 | 8.9 |
| 7 | 658 | 75.7 | 16.7 | 7.6 | 0.263 | 18.6 | 11.1 | 8.9 | 10.0 |
| 8 | 648 | 72.3 | 18.5 | 9.3 | 0.269 | 20.5 | 6.7 | 7.6 | 7.1 |
| 9 | 648 | 72.3 | 19.7 | 8.0 | 0.258 | 21.8 | 13.3 | 6.5 | 9.3 |
| 10 | 642 | 72.9 | 18.7 | 8.4 | 0.265 | 20.1 | 13.3 | 9.3 | 11.1 |
| 11 | 598 | 78.3 | 13.3 | 8.4 | 0.276 | 20.5 | 9.4 | 4.8 | 6.7 |
| 12 | 618 | 75.8 | 12.9 | 11.3 | 0.302 | 21.7 | 13.9 | 4.8 | 8.1 |
| 15 | 40 | 0.0 | 50.0 | 50.0 | | 8.5 | 0.0 | 0.0 | 0.0 |
| 16 | 568 | 82.5 | 14.0 | 3.5 | | 24.5 | 40.0 | 25.0 | 31.6 |

| | | | | MD Elong | CD Elong |
|---|---|---|---|---|---|
| | Intake | Intake | Intake | Ld @ 50% | Ld @ 50% |
| EXAMPLE | Insult 1 | Insult 2 | Insult 3 | (A) (cycle- | (A) (cycle- |
| # | (ml/s) | (ml/s) | (ml/s) | 1) (gf/in) | 1) (gf/in) |
| 1 | 0.33 | 1.22 | 1.81 | 307.1 | 334.7 |
| 2 | 0.50 | 1.06 | 1.39 | 299.9 | 337.2 |
| 3 | 1.01 | 1.60 | 1.55 | 259.9 | 288.6 |
| 4 | 0.19 | 0.25 | 0.21 | 343.6 | 392.525 |
| 5 | 0.11 | 0.18 | 0.19 | 319.4 | 372.675 |
| 6 | 0.40 | 0.50 | 0.39 | 252.5 | 260.95 |
| 7 | 1.43 | 0.76 | 0.64 | 192.5 | 249.725 |
| 8 | 0.86 | 0.85 | 1.01 | 155.7 | 260.75 |
| 9 | 1.00 | 0.89 | 1.04 | 133 | 193.05 |
| 10 | 0.94 | 0.95 | 1.19 | 145.1 | 259.975 |
| 11 | 0.74 | 0.81 | 0.96 | 149.2 | 224.175 |
| 12 | 0.73 | 0.78 | 0.87 | 212.9 | 315.875 |
| 15 | | | | | |
| 16 | | | | | |

TABLE 3

| | | % CD | % MD | ZD | MD | CD | ZD |
|---|---|---|---|---|---|---|---|
| | | growth | growth | Tensile | Average | Average | Tensile/MD |
| EXAMPLE | Stratified | (Full | (Full | Strength | Modulus | Modulus | Modulus |
| # | (yes = 1) | Pad) | Pad) | (MPa) | (MPa) | (MPa) | (MPa/MPa) |
| 1 | 0 | 33.3 | 39.3 | 0.0068 | 0.4110 | 0.4264 | 0.0165 |
| 2 | 0 | 16.7 | 30.6 | 0.0063 | 0.2553 | 0.2960 | 0.0246 |
| 3 | 0 | 16.7 | 24.0 | 0.0092 | 0.2794 | 0.2015 | 0.0330 |
| 4 | 1 | 14.4 | 17.9 | 0.0022 | 0.2923 | 0.2285 | 0.0055 |
| 5 | 1 | 10.0 | 15.2 | 0.0024 | 0.3096 | 0.2608 | 0.0095 |
| 6 | 1 | 6.7 | 11.8 | 0.0008 | 0.1657 | 0.1329 | 0.0059 |
| 7 | 1 | 11.1 | 8.9 | 0.0006 | 0.0996 | 0.1445 | 0.0063 |
| 8 | 1 | 6.7 | 7.6 | 0.0003 | 0.0793 | 0.0958 | 0.0036 |
| 9 | 1 | 13.3 | 9.3 | 0.0004 | 0.0610 | 0.0971 | 0.0059 |
| 11 | 1 | 9.4 | 4.8 | 0.0004 | 0.0738 | 0.1113 | 0.0049 |
| 12 | 1 | 13.9 | 4.8 | 0.0005 | 0.1097 | 0.1687 | 0.0049 |
| 15 | 1 | 0.0 | 0.0 | 0.0192 | 0.0303 | 0.2098 | 0.6341 |
| 16 | 1 | 40.0 | 25.0 | 0.0007 | 0.0549 | 0.0900 | 0.0121 |

It can be observed from Table 2 and Table 3 that in comparison to conventional stretchable absorbent composites, the composites of the present invention have substantially lower wet growth as seen in the individual MD and CD Growth values as well as the Geometric Mean Growth values. For instance, the homogeneous non-stratified Comparative Example 1 has a Geometric Mean Growth of 36.2% while Example 8 of the invention has a Geometric Mean Growth of 7.1%.

As illustrated in Table 3, the ZD Tensile Strength is substantially decreased for absorbents of the inventions, allowing for greater swelling in the ZD. As described earlier, the relationship between MD Modulus and ZD Tensile also governs the relative swelling between the X-Y planes and the ZD. For absorbents of the invention, the MD Modulus is at least 75 times greater than the ZD tensile strength as measured by the MD Modulus Test and the ZD Tensile Test, respectively. For example, this ratio is 61 for Comparative Example 1 versus a ratio of 278 for Example 8 of the invention.

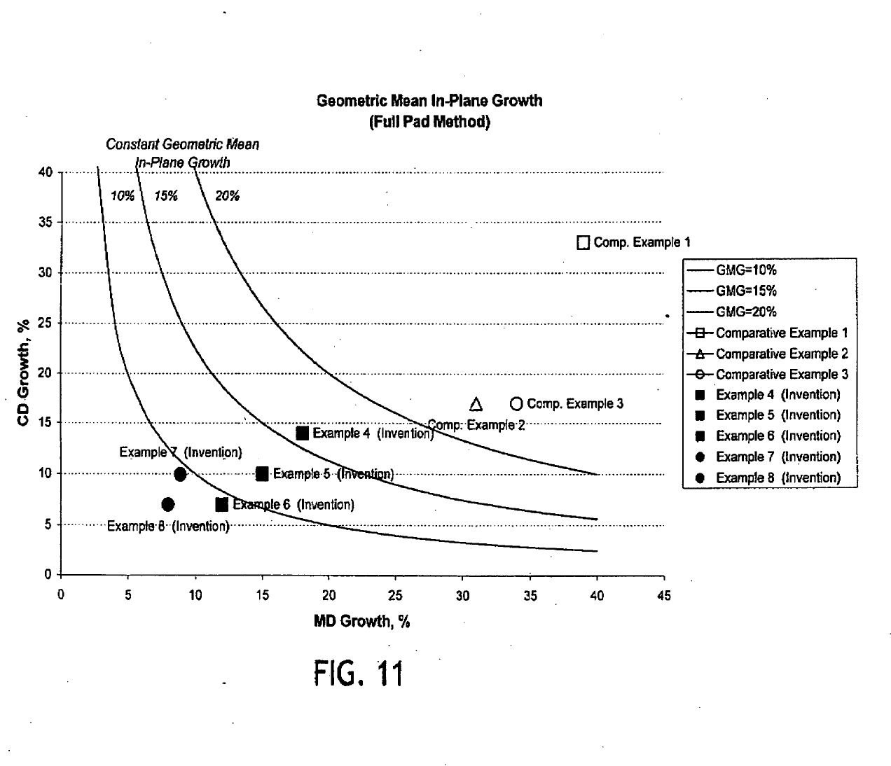

In addition, attention is drawn to FIG. 11 which demonstrates the stability of a composite of the present invention in the x-y plane (MD-CD plane) as compared to a conventional stretchable absorbent composite. It can be seen that examples characterizing the invention have significantly lower MD and CD wet growth compared to the Comparative Examples. Also superimposed on the graph are lines of constant Geometric Mean Growth for reference. The composites of the invention have less than 20% Geometric Mean Growth, some have less than 15% and other have less than 10% wet growth, as seen in FIG. 11 and the Tables.

Examples 15 and 16 demonstrate the properties of the individual regions.

Example 15 represents a low capacity region only, and Example 16 represents a high capacity region only. A stratified absorbent composite could have a structure as seen in Table 4 below. When tested, Example 16 was found to exhibit substantial Geometric Wet Growth of 31.6% despite having a relatively low elastomeric polymer content of 3.5%. This illustrates the synergistic effect of having both regions present in a stratified configuration of the invention.

TABLE 4
ABSORBENCY CONTRIBUTIONS BY EACH REGION

| | | | Component | | Composite | |
|---|---|---|---|---|---|---|
| | | | Saturated | Component | Component | Total |
| EXAMPLE | | Location if in | Capacity | Basis Weight, | Content | Capacity, |
| # | Description | a Composite | g/g | gsm | % | g fluid |
| Example 15 | Low Capacity | 1^{st} Outer | 8.5 | 40 | 6.2% | 11.3 |
| | Region | region | | | | |
| Example 16 | High Capacity | Middle region | 24.5 | 568 | 87.7% | 460.9 |
| | Region | | | | | |
| Example 15 | Low Capacity | 2^{nd} Outer | 8.5 | 40 | 6.2% | 11.3 |
| | Region | region | | | | |
| | Total | | | 648 | 100.0% | 483.4 |

In Table 4, the individual regions of Examples 15 and 16 are used to demonstrate how the test methods cited here can easily differentiate the high and low absorbent regions. Incidently, the middle high capacity region of Example 8 has a composition similar to Example 16 and the outer low capacity regions of Example 8 have compositions similar to Example 15.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

### Test Procedures

Saturated Capacity Test

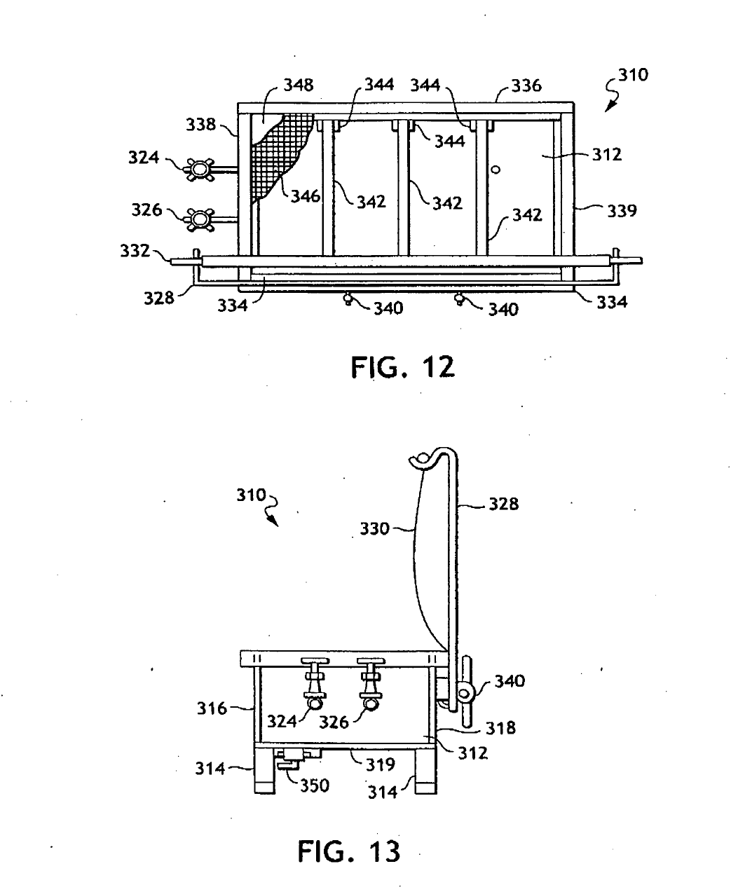

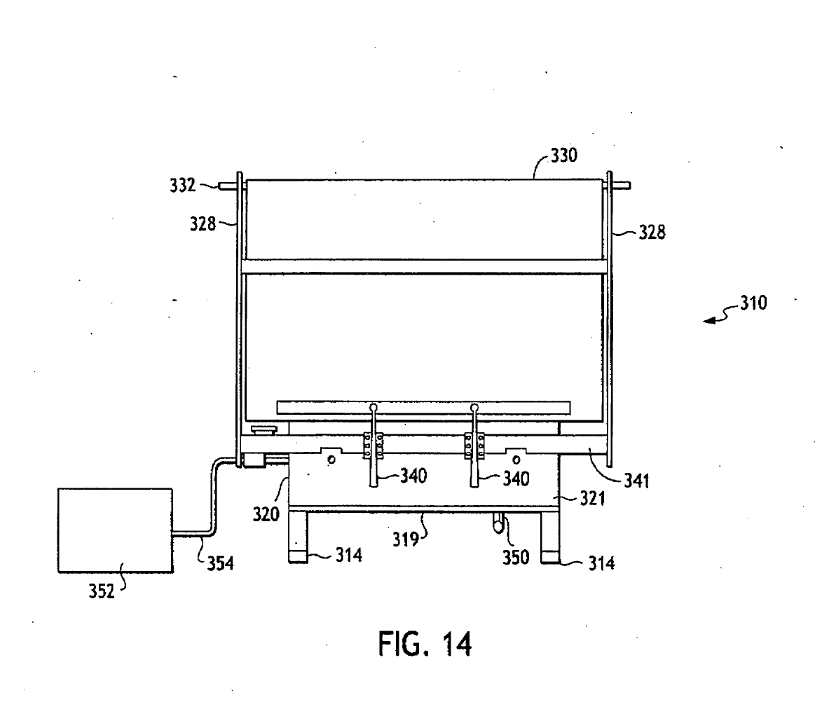

Saturated Capacity is determined using a Saturated Capacity (SAT CAP) tester with a Magnahelic vacuum gage and a latex dam, comparable to the following description. Referring to FIGS. 12-14, a Saturated Capacity tester vacuum apparatus 310 comprises a vacuum chamber 312 supported on four leg members 314. The vacuum chamber 312 includes a front wall member 316, a rear wall member 318, and two side walls 320 and 321. The wall members are sufficiently thick to withstand the anticipated vacuum pressures, and are constructed and arranged to provide a chamber having outside dimensions measuring 23.5 inches (59.7 cm) in length, 14 inches (35.6 cm) in width and 8 inches (20.3 cm) in depth.

A vacuum pump (not shown) operably connects with the vacuum chamber 312 through an appropriate vacuum line conduit and a vacuum valve 324. In addition, a suitable air bleed line connects into the vacuum chamber 312 through an air bleed valve 326. A hanger assembly 328 is suitably mounted on the rear wall 318 and is configured with S-curved ends to provide a convenient resting place for supporting a latex dam sheet 330 in a convenient position away from the top of the vacuum apparatus 310. A suitable hanger assembly can be constructed from 0.25 inch (0.64 cm) diameter stainless steel rod. The latex dam sheet 330 is looped around a dowel member 332 to facilitate grasping and to allow a convenient movement and positioning of the latex dam sheet 330. In the illustrated position, the dowel member 332 is shown supported in a hanger assembly 328 to position the latex dam sheet 330 in an open position away from the top of the vacuum chamber 312.

A bottom edge of the latex dam sheet 330 is clamped against a rear edge support member 334 with suitable securing means, such as toggle clamps 340. The toggle clamps 340 are mounted on the rear wall member 318 with suitable spacers 341 which provide an appropriate orientation and alignment of the toggle clamps 340 for the desired operation. Three support shafts 342 are 0.75 inches in diameter and are removably mounted within the vacuum chamber 312 by means of support brackets 344. The support brackets 344 are generally equally spaced along the front wall member 316 and the rear wall member 318 and arranged in cooperating pairs. In addition, the support brackets 344 are constructed and arranged to suitably position the uppermost portions of the support shafts 342 flush with the top of the front, rear and side wall members of the vacuum chamber 312. Thus, the support shafts 342 are positioned substantially parallel with one another and are generally aligned with the side wall members 320 and 321. In addition to the rear edge support member 334, the vacuum apparatus 310 includes a front support member 336 and two side support members 338 and 339. Each side support member measures about 1 inch (2.5 cm) in width and about 1.25 inches (3.2 cm) in height. The lengths of the support members are constructed to suitably surround the periphery of the open top edges of the vacuum chamber 312, and are positioned to protrude above the top edges of the chamber wall members by a distance of about 0.5 inches.

A layer of egg crating type material 346 is positioned on top of the support shafts 342 and the top edges of the wall members of the vacuum chamber 312. The egg crate material extends over a generally rectangular area measuring 23.5 inches (59.7 cm) by 14 inches (35.6 cm), and has a depth measurement of about 0.38 inches (1.0 cm). The individual cells of the egg crating structure measure about 0.5 inch square, and the thin sheet material comprising the egg crating is composed of a suitable material, such as polystyrene. For example, the egg crating material can be McMaster-Carr Supply Catalog No. 1624K 14 translucent diffuser panel material (available from McMaster-Carr Supply Company, having a place of business in Atlanta, Ga. U.S.A.). A layer of 6 mm (0.24 inch) mesh TEFLON-coated screening 348 (available from Eagle Supply and Plastics, Inc., having a place of business in Appleton, Wis., U.S.A.) which measures 23.5 inches (59.7 cm) by 14 inches (35.6 cm), is placed on top of the egg crating material 346.

A suitable drain line and a drain valve 350 connect to the bottom plate member 319 of the vacuum chamber 312 to provide a convenient mechanism for draining liquids from the vacuum chamber 312. The various wall members and support members of the vacuum apparatus 310 may be composed of a suitable non-corroding, moisture-resistant material, such as polycarbonate plastic. The various assembly joints may be affixed by solvent welding and/or fasteners, and the finished assembly of the tester is constructed to be water-tight. A vacuum gauge 352 operably connects through a conduit into the vacuum chamber 312. A suitable pressure gauge is a Magnahelic differential gauge capable of measuring a vacuum of 0-100 inches of water, such as a No. 2100 gauge available from Dwyer Instrument Incorporated (having a place of business in Michigan City, Ind., U.S.A.) The dry product or other absorbent structure is weighed and then placed in excess 0.9% NaCl saline solution, submerged and allowed to soak for twenty (20) minutes. After the twenty (20) minute soak time, the absorbent structure is placed on the egg crate material and mesh TEFLON-coated screening of the Saturated Capacity tester vacuum apparatus 310. The latex dam sheet 330 is placed over the absorbent structure(s) and the entire egg crate grid so that the latex dam sheet 330 creates a seal when a vacuum is drawn on the vacuum apparatus 310. A vacuum of 0.5 pounds per square inch (psi) is held in the Saturated Capacity tester vacuum apparatus 310 for five minutes. The vacuum creates a pressure on the absorbent structure(s), causing drainage of some liquid. After five minutes at 0.5 psi vacuum, the latex dam sheet 330 is rolled back and the absorbent structure(s) are weighed to generate a wet weight.

The overall capacity of each absorbent structure is determined by subtracting the dry weight of each absorbent from the wet weight of that absorbent, determined at this point in the procedure. The 0.5 psi Saturated Capacity, or Saturated Capacity, of the absorbent structure is determined by the following formula:
Saturated Capacity=(wet weight−dry weight)/dry weight;
wherein the Saturated Capacity value has units of grams of fluid/gram of absorbent. For Saturated Capacity, a minimum of three specimens of each sample should be tested and the results averaged. If the absorbent structure has low integrity or disintegrates during the soak or transfer procedures, the absorbent structure can be wrapped in a containment material such as paper toweling, for example SCOTT paper towels manufactured by Kimberly-Clark Corporation, having a place of business in Neenah, Wis., U.S.A. The absorbent structure can be tested with the overwrap in place and the capacity of the overwrap can be independently determined and subtracted from the wet weight of the total wrapped absorbent structure to obtain the wet absorbent weight.
Fluid Intake Rate Test

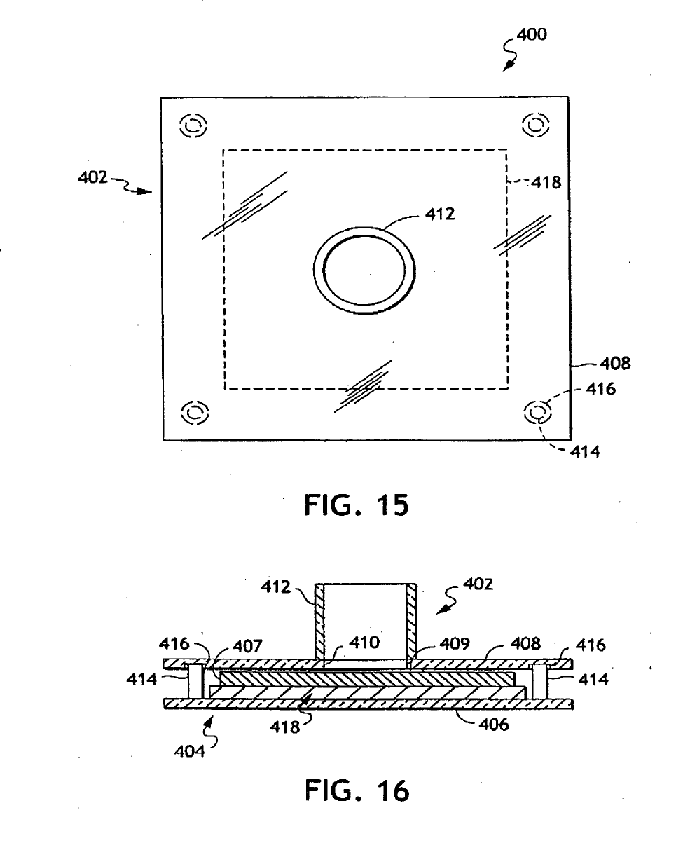

The Fluid Intake Rate (FIR) Test determines the amount of time required for an absorbent structure to take in (but not necessarily absorb) a known amount of test solution (0.9 weight percent solution of sodium chloride in distilled water at room temperature). A suitable apparatus for performing the FIR Test is shown in FIGS. 15 and 16 and is generally indicated at 400. The test apparatus 400 comprises upper and lower assemblies, generally indicated at 402 and 404 respectively, wherein the lower assembly comprises a generally 7 inch by 7 inch (17.8 cm×17.8 cm) square lower plate 406 constructed of a transparent material such as PLEXIGLAS (available from Degussa AG, having a place of business in Dusseldorf, Germany) for supporting the absorbent sample during the test and a generally 4.5 inch by 4.5 inch (11.4 cm x 11.4 cm) square platform 418 centered on the lower plate 406.

The upper assembly 402 comprises a generally square upper plate 408 constructed similar to the lower plate 406 and having a central opening 410 formed therein. A cylinder (fluid delivery tube) 412 having an inner diameter of about one inch (2.5 cm) is secured to the upper plate 408 at the central opening 410 and extends upward substantially perpendicular to the upper plate. The central opening 410 of the upper plate 408 should have a diameter at least equal to the inner diameter of the cylinder 412 where the cylinder 412 is mounted on top of the upper plate 408. However, the diameter of the central opening 410 may instead be sized large enough to receive the outer diameter of the cylinder 412 within the opening so that the cylinder 412 is secured to the upper plate 408 within the central opening 410.

Pin elements 414 are located near the outside corners of the lower plate 406, and corresponding recesses 416 in the upper plate 408 are sized to receive the pin elements 414 to properly align and position the upper assembly 402 on the lower assembly 404 during testing. The weight of the upper assembly 402 (e.g., the upper plate 408 and cylinder 412) is approximately 360 grams to simulate approximately 0.11 pounds/square inch (psi) pressure on the absorbent sample during the FIR Test.

To run the FIR Test, an absorbent sample 407 being three inches (7.6 cm) in diameter is weighed and the weight is recorded in grams. The sample 407 is then centered on the platform 418 of the lower assembly 404. The upper assembly 402 is placed over the sample 407 in opposed relationship with the lower assembly 404, with the pin elements 414 of the lower plate 406 seated in the recesses 416 formed in the upper plate 408 and the cylinder 412 is generally centered over the sample 407. Prior to running the FIR test, the aforementioned Saturated Capacity Test is measured on the sample 407. Thirty percent (30%) of the saturation capacity is then calculated by multiplying the mass of the dry sample (grams) times the measured saturated capacity (gram/gram) times 0.3; e.g., if the test sample has a saturated capacity of 20 g of 0.9% NaCl saline test solution/g of test sample and the three inch (7.6 cm) diameter sample 407 weighs one gram, then 6 grams of 0.9% NaCl saline test solution (referred to herein as a first insult) is poured into the top of the cylinder 412 and allowed to flow down into the absorbent sample 407. A stopwatch is started when the first drop of solution contacts the sample 407 and is stopped when the liquid ring between the edge of the cylinder 412 and the sample 407 disappears. The reading on the stopwatch is recorded to two decimal places and represents the intake time (in seconds) required for the first insult to be taken into the absorbent sample 407.

A time period of fifteen minutes is allowed to elapse, after which a second insult equal to the first insult is poured into the top of the cylinder 412 and again the intake time is measured as described above. After fifteen minutes, the procedure is repeated for a third insult. An intake rate (in milliliters/second) for each of the three insults is determined by dividing the amount of solution (e.g., six grams) used for each insult by the intake time measured for the corresponding insult.

At least three samples of each absorbent test are subjected to the FIR Test and the results are averaged to determine the intake rate.

Full Pad Growth Test

The Saturated Capacity test apparatus and operating procedure is described in detail in the above Saturated Capacity Test section (also referred to as “SatCap”) and can be applied to most samples that can adequately fit on the grid and be sealed with the rubber sheet, including the testing of Full Pad absorbent samples as described herein. Z-direction thickness testing of the dry and wet pads is done using a thickness gauge with a 3″ circular foot exerting a 0.05 psi pressure.

For absorbent materials of comparative examples and the invention, a die was used to cut out hour-glass shaped pads measuring 14 inches (35.6 cm) long in the MD and 3 inches (7.6 cm) in the CD representing a typical pad shape often found in diapers. These were the “dry lengths” used for the MD and CD Wet Growth calculation. Alternatively, the dry measurements of the full pad can be measured with a ruler. The narrowest dimension of 3 inch (7.6 cm) approximates the crotch area in a typical diaper or training pant.

To insure retention of all superabsorbent and to facilitate handling of the saturated pad, each sample was wrapped in an absorbent paper towel during testing. The wrap should approximate a length of 40 inches (102 cm) and width of 11 inches (27.9 cm), or of sufficient size to wrap the absorbent pad. SCOTT Towel Mega Roll or equivalent can be used. The Saturation Capacity of the paper towels alone was measured and recorded using the same Saturation Capacity procedure and settings (set for 0.05 psi or 14 inches water for 5 minutes).The contribution of the dry and wet weights of the paper towels was removed from the final results by calculation.

The MD length and CD length of the dry absorbent pad is measured with a ruler and recorded to the nearest 0.1 inch (0.25 cm). The Z-direction dry thickness is measured as well. Next, each full pad sample is placed on and wrapped in a length of four unseparated paper towel sheets as previously described. The edges of the towel are then carefully folded over the top of the pad. The pad should remain planar and extended to full width and length (not folded over). The towel portion is previously weighed dry and labeled. The dry weight of the full pad and towel assembly is then weighted and recorded. The assembly is then placed on a stiff mesh screen capable of supporting the wet weight of the assembly.

Each assembly with support screen is then immersed in 0.9% saline solution for 20 minutes. Still supported, the assembly is removed from the saline and placed (still wrapped with the paper towel) on the retention capacity box screen and covered with the rubber membrane. The box vacuum is set for 0.05 psi (14 inches water) for 5 minutes.

Each assembly is then removed, weighed and the wet weight recorded. The towel wrap is then removed and the full pad placed on a flat surface while being careful to avoid folds, misalignment and stretching. The length and width of the wet pad is measured with a ruler to the nearest 0.1 inch. These are the “wet lengths.” The Z-direction wet thickness is measured as well. Three full pad samples are tested for each code.

The MD, CD and ZD Wet Growth values as calculated as follows:
% CD Growth=(CD Pad Wet Length−CD Pad Dry Length)/CD Pad Dry Length×100
% MD Growth=(MD Pad Wet Length−MD Pad Dry Length)/MD Pad Dry Length×100
% ZD Growth=(ZD Pad Wet Length−ZD Pad Dry Length)/ZD Pad Dry Length×100
The results are reported as average values of % CD Growth, % MD Growth, and % ZD Growth.
The Geometric Mean Growth can then be computed as follows:
Geometric Mean Growth, %=SQRT(% CD Growth×% MD Growth)

A Full Pad Saturated Capacity (also referred to as “SatCap”) can be computed as well, applying the formula cited in the “Saturated Capacity Test” procedure.

Elongation Test

The Elongation Test measures how much tension a sample can exert when stretched to a given level (50% in this case).

The absorbent composite is cut into 3-inch (7.62 cm) by 7-inch (17.78 cm) specimens. A Constant Rate of Extension (CRE) tensile tester: MTS tensile tester model Alliance RT/1 or equivalent, available from MTS Systems Corporation, Research Triangle Park, N.C., U.S.A. is used to measure Elongation. A substantially equivalent testing device may optionally be employed. Each specimen is mounted onto the equipment vertically with two clamps and the locations of the clamps are marked on the specimen. The distance between the two clamps (Lₒ) is 2 inches (5.1 cm). The specimen is stretched by moving the upper clamp upward at a rate of 500 mm/min and is held for 5 seconds at the predetermined length of extension (Lₑ=1.5×Lₒ) and the load is recorded. This is the elongation load at 50% extension. The elongation value is the elongation load divided by three and is reported in units of grams-force per inch (g_{f}/in.). The upper clamp is returned to the original position and the specimen is free to retract. For each absorbent composite, test specimens are prepared and subjected to Elongation testing with respect to both the machine direction (MD) and the cross-machine direction (CD) of the absorbent composite.

ZD Tensile Test

The Z-direction tensile strength (ZD Tensile) is measured by pulling a 7.6 cm diameter die cut sample apart with two 5.1 cm wide by 5.1 cm long pieces of double sided tape Type 406 (available from 3M Corporation,) adhered to either side of the specimen in the tensile frame (MTS tensile tester model Alliance RT/1 or equivalent, available from MTS Systems Corporation, Research Triangle Park, N.C., U.S.A.) in a TAPPI conditioned Laboratory (22° C., 50% RH). The sample was pulled apart between two parallel, circular platens. The lower platen with a diameter of 8.9 cm, and the upper platen with a diameter of 5.7 cm. The tape was adhered to both sides of the sample by first attaching the tape to the upper and lower plattens so that the comers of each piece were aligned with the piece opposite, and then placing the sample on the lower tape so the sample was centered on the tape and then applying a 200 lb_{f} (90.7 kg_{f}) compression load using the frame. The platens of the tester were moved apart at 1.3 cm/min up to a separation of 2.5 cm. The frame recorded the load (in Newtons) versus displacement during the test and the peak tensile load is recorded. The ZD Tensile (in MPa) is calculated by dividing the peak load (in Newtons) by the 2581 mm² area of the tapes pulling the specimen apart. The test is repeated three times for each code and the ZD Tensile is reported as the average of the 3 measurements.

MD Modulus Test

Sample Preparation

Three samples of the test specimen are subjected to MD Slope Test, and the results for each set of three samples are averaged. Each sample are approximately 2 inches (50.8 mm) wide by at least 3 inches (76.2 mm) long. The samples are cut from the midline of the specimen with the 3 inch dimension aligned with the machine direction of the sample.

Test Apparatus and Materials

The following test apparatus and materials are used to conduct the MD Slope Test.

1) Constant Rate of Extension (CRE) tensile tester: MTS tensile tester model Alliance RT/1 or equivalent, available from MTS Systems Corporation, Research Triangle Park, N.C., U.S.A.

2) Load cells: A suitable cell selected so that the majority of the peak load values fall between the manufacturer's recommended ranges of the load cell's full scale value. Load cell Model 100N available from MTS Systems Corporation is preferred.

3) Operating software and data acquisition system: MTS TESTWORKS for Windows software version 4, available form MTS Systems Corporation.

4) Grips: pneumatic-action grips, top and bottom, identified as part number 2712-003 available from Instron Corporation, Canton, Mass., U.S.A.

5) Grip faces: 25 mm by 100 mm.

Test Conditions

Reasonable ambient conditions should be used for sample testing, such as 73 +/−20° F. (about 23° C.) and a relative humidity of 50 +/−2%. If the samples are stored under substantially different conditions, the samples should be measured after they equilibrate to laboratory conditions.

The instruments used should be calibrated as described in the manufacturer's instructions for each instrument.

The tensile tester conditions are as follows:

Break sensitivity: 60%

Break threshold: 200 grams-force

Data acquisition rate: 100 Hz

Preload?: No

Slowdown extension: 0 mm

Test speed: 254 mm/min.

Full scale load: 10,000 grams-force

Gage length: 2 inches (50.8 mm)

Test Method

Calibrate the load cell using the TESTWORKS software at the beginning of each work session. Using the tensile frame pushbutton controls for cross-head position, move the grips to provide a gage length (distance between grips) of 2 inches (50.8 mm). Calibrate the software to this initial gage length. Place the sample to be tested lengthwise so that it is centered between the grips, held in a centered position within each grip, and oriented correctly (e.g., with the widthwise dimension running transverse to the length between the grips), e.g., with the vertical (e.g., side) edges of the sample perpendicular to the grip faces. Close the grips on the sample, holding the sample in such a way as to minimize slack in the sample without placing the sample under tension.

Ensure that the load at this point is less than ±3.3 grams per inch of sample width. If the load is greater than 3.3 grams per inch width, release the lower grip and zero the load cell. Re-close the lower grip, again ensuring that the sample is neither under tension nor buckled with excessive slack. Continue checking the starting load and following the above procedure until the starting load is within the desired range.

Run the test using the above parameters by clicking on the RUN button. When the test is complete, save the data to a sample file. Remove the sample from the grips. Run the above procedures for the remaining samples of a given specimen. The data for all samples should be saved to a single file.

Use the Testworks4 software to calculate the average slope of loading between 70 and 157 g_{f}. This average slope is the MD Slope and is reported in units of “g_{f}/2”.

Calculation

The MD Modulus is calculated as:

$$MDModulus = \frac{MDSlope \times 0.0098}{Caliper \times 50.8}$$

Where MD Modulus is the modulus in MPa (N/mm²), MD Slope is calculated as described above, Caliper is the thickness of the sample measured under a load of 0.05 psi, 0.0098 is the number of Newtons per gram, and 50.8 is the width of the sample in mm. The Caliper is measured using a bulk meter with a 7.6 cm diameter foot attached to a displacement meter (Mitutoyo or similar) with the foot weighted so that it exerts a 345 Pa (0.05 psi) pressure on the sample. The CD Modulus is measured following the same procedure on a sample that has its testing axis aligned in the CD.
Geometric Mean Growth Test and Geometric Mean Modulus Test

The geometric mean of either the growth or the modulus is defined by the following calculation:
E_{GM}=\sqrt{E_{CD}E_{MD}}
Where E_{CD} and E_{MD} are the properties (i.e., growth or modulus) measured in the cross-machine direction and machine direction, respectively.

1. An absorbent composite comprising a low capacity region and a high capacity region in planar relationship to the low capacity region;
wherein the high capacity region comprises between 1% and 10% by weight elastomeric polymer fibers and between 60% and 98% by weight superabsorbent material; and
wherein the low capacity region comprises at least 10% by weight elastomeric polymer fibers and less than 10% by weight superabsorbent material.

2. The absorbent composite of claim 1 wherein the elastomeric fibers of high capacity region and of the low capacity region are intermingled.

3. The absorbent composite of claim 1 further comprising an additional region.

4. The absorbent composite of claim 3 wherein the high capacity region is positioned between the low capacity region and the additional region.

5. The absorbent composite of claim 4 having a perimeter area and a central area, wherein the high capacity region is positioned only in the central area of the absorbent composite.

6. The absorbent composite of claim 5 wherein the fibers of the low capacity region and the additional region located in the perimeter area are intermingled.

7. The absorbent composite of claim 1 wherein the high capacity region comprises between 1% and 5% by weight elastomeric polymer fibers.

8. The absorbent composite of claim 1 wherein the high capacity region comprises between 1% and 3% by weight elastomeric polymer fibers.

9. The absorbent composite of claim 1 wherein the low capacity region comprises substantially meltblown elastomeric polymer fibers.

10. The absorbent composite of claim 1 wherein the elastomeric polymer fibers are substantially continuous.

11. The absorbent composite of claim 1 wherein the elastomeric polymer fibers have an average fiber diameter between 5 μm and 50 μm.

12. The absorbent composite of claim 1 wherein the elastomeric polymer fibers have an average fiber diameter between 10 μm and 25 μm.

13. The absorbent composite of claim 1 wherein the low capacity region further comprises between 10% and 90% by weight cellulosic fiber.

14. The absorbent composite of claim 1 wherein the low capacity region further comprises between 30% and 70% by weight cellulosic fiber.

15. The absorbent composite of claim 1 wherein the low capacity region has a basis weight of between 5 and 100 gsm.

16. The absorbent composite of claim 1 wherein the low capacity region has a basis weight of between 10 and 50 gsm.

17. The absorbent composite of claim I wherein at least one of the low capacity region and the high capacity region has been treated to be hydrophilic.

18. The absorbent composite of claim 1 wherein the high capacity region farther comprises 20% or less by weight cellulosic fiber.

19. The absorbent composite of claim 1 wherein the high capacity region has a basis weight of between 25 and 1000 gsm.

20. The absorbent composite of claim 1 wherein the superabsorbent material comprises a coating to improve attachment of the superabsorbent material to the elastomeric polymer fibers when compared to an uncoated superabsorbent material.

21. The absorbent composite of claim 20 wherein the coating includes at least one material selected from modified maleated propylene, polyvinyl alcohol, polyethylene oxide, polypropylene oxide, ethylene oxide-propylene oxide copolymer, polyethylene glycol, polypropylene glycol, ethylene glycol-propylene glycol copolymer, modified polysaccharides, such as hydroxypropyl cellulose, methyl cellulose, methyl ethyl cellulose, polyethylene imine, or combinations thereof.

22. The absorbent composite of claim 1 having an absorbency of at least 16 g/g as measured by the Saturated Capacity Test.

23. The absorbent composite of claim 1 having a fluid intake rate of at least 0.4 ml/sec as measured by the Fluid Intake Rate Test.

24. The absorbent composite of claim 1 having a Geometric Mean Growth of 20% or less as measured by the Full Pad Growth Test.

25. The absorbent composite of claim 1 having a Geometric Mean Growth of 10% or less as measured by the Full Pad Growth Test.

26. The absorbent composite of claim 1 having an MD Modulus at least 75 times greater than the ZD tensile strength as measured by the MD Modulus Test and the ZD Tensile Test, respectively.

27. The absorbent composite of claim 1 having a geometric mean modulus of less than 1 MPa as measured by the Geometric Mean Modulus Test.

28. The absorbent composite of claim 1 having an MD elongation at 50% extension of at least 100 g_{f}/inch as measured by the Elongation Test.

29. The absorbent composite of claim 1 having a CD elongation at 50% extension of at least 100 g_{f}/inch as measured by the Elongation Test.

30. The absorbent composite of claim 1 further including at least one of a liquid-permeable topsheet and a backsheet; wherein the high capacity region and the low capacity region are disposed between the topsheet and backsheet.
